(19) United States
(12) Patent Application Publication
(10) Pub. No.: US 2021/0177895 A1
(43) Pub. Date: Jun. 17, 2021

(54) METHODS OF MODULATING M2 MACROPHAGE POLARIZATION AND USE OF SAME IN THERAPY

(71) Applicant: Yeda Research and Development Co. Ltd., Rehovot (IL)

(72) Inventors: Ido AMIT, Rehovot (IL); Merav COHEN, Rehovot (IL); Amir GILADI, Rehovot (IL)

(73) Assignee: Yeda Research and Development Co. Ltd., Rehovot (IL)

(21) Appl. No.: 17/183,593

(22) Filed: Feb. 24, 2021

Related U.S. Application Data
(63) Continuation of application No. PCT/IL2019/050939, filed on Aug. 21, 2019.
(60) Provisional application No. 62/722,196, filed on Aug. 24, 2018.

(51) Int. Cl. A61K 35/15; C12N 5/0787

(52) U.S. Cl. CPC A61K 35/15; C12N 2501/125; C12N 2501/2333; C12N 5/0642

(57) ABSTRACT

A method of treating a disease or disorder that can benefit from increasing an M2/M1 macrophage ratio in a subject in need thereof is provided. The method comprising: (a) culturing basophils in the presence of IL33 and/or GM-SCF; and (b) administering to the subject a therapeutically effective amount of the basophils following the culturing, thereby treating the disease or disorder that can benefit from increasing an M2/M1 macrophage ratio in the subject.

### RELATED APPLICATIONS

This application is a Continuation of PCT Patent Application No. PCT/IL2019/050939 having international filing date of Aug. 21, 2019, which claims the benefit of priority under 35 USC § 119(e) of US Provisional Patent Application No. 62/722,196, filed on Aug. 24, 2018. The contents of the above applications are all incorporated by reference as if fully set forth herein in their entirety.

### FIELD AND BACKGROUND OF THE INVENTION

The present invention, in some embodiments thereof, relates to methods of modulating M2 macrophage polarization and use of same in therapy.

Mammalian tissues consist of diverse cell types that include: fibroblasts, epithelial, endothelial and immune lineages. Tissue formation during embryonic development requires the coordinated function and crosstalk between distinct cell types, in specific environmental contexts. Development of the lung into specialized committed cell types is a highly regulated process, characterized by unique pathways and functional properties. In parallel, cells of the immune system migrate from hematopoietic sites to the lung, in order to establish an active immune compartment that interacts with stromal cells, and influences tissue differentiation, growth and function.

The mammalian lung is the central respiratory organ, featuring a diverse set of specialized cell types. Gas exchange in the lung occurs in the alveoli, which are composed of specialized epithelial cells: the alveolar type (AT) 1 cells that mediate gas exchange, and AT2 cells that secrete surfactant and maintain the surface tension of the lungs (Whitsett and Alenghat, 2015). Alveolar epithelial cells branch from their mutual progenitor between the canalicular (E16.5) and saccular (E18.5) stages, resulting in dramatic changes in morphology and gene expression (Treutlein et al., 2014). Another major cell type is the alveolar macrophages (AM), which clear surfactant from the alveolar space, and act as important immune modulators, suppressing unwanted immune responses in the lungs (Hussell and Bell, 2014). AM originate from fetal liver embryonic precursors and are self-maintaining, with no contribution from the adult bone marrow (Epelman et al., 2014; Hashimoto et al., 2013; Murphy et al., 2008; Shibata et al., 2001). The first wave of lung macrophages appears at embryonic day 12.5 (E12.5), followed by a second wave stemming from fetal-liver derived monocytes, which continues its differentiation axis during alveolarization into mature AM (Ginhoux, 2014; Ginhoux and Jung, 2014; Hoeffel and Ginhoux, 2018; Kopf et al., 2015; Tan and Krasnow, 2016).

The immune response in each tissue, and the lung in particular, must be tightly regulated and adapted to its requirements, as aberrant immune activation may cause tissue damage and pathologies including chronic inflammation, fibrosis and autoimmune responses. Hence, each tissue is equipped with a unique signaling environment that interacts with the immune compartment and shapes the gene expression and chromatin landscapes of the cells (Butovsky et al., 2014; Cipolletta et al., 2015; Cohen et al., 2014; Greter et al., 2012; Hussell and Bell, 2014; Lavin et al., 2014; Okabe and Medzhitov, 2014; Panduro et al., 2016; Yu et al., 2017). In the lung context, AM exhibit a tissue specific phenotype, evident by their gene expression and function (Gautier et al., 2012; Guilliams et al., 2013b; Kopf et al., 2015; Lavin et al., 2014). There is a major gap in our understanding of the dynamic signaling during the alveolarization process, as attempts to grow AM ex vivo have not been successful (Fejer et al., 2013). Lung macrophage development and maturation was shown to be dependent on different growth and differentiation cues transmitted from epithelial cells (mainly AT2), innate lymphocytes (ILC) and the AM themselves (de Kleer et al., 2016; Guilliams et al., 2013a; Saluzzo et al., 2017; Yu et al., 2017). The function and crosstalk of other lung resident immune and non-immune cell types in the lung is currently much less understood.

Basophils are thought to be short-lived granulocytic cells, characterized by the presence of lobulated nuclei and secretory granules in the cytoplasm. They complete their maturation in the bone-marrow, before they enter and patrol the bloodstream. Under pathological conditions, such as parasite infection and allergic disorders, basophils are recruited and invade tissue parenchyma (Min et al., 2004; Mukai et al., 2005; Oh et al., 2007), and their major function has been mainly attributed to induction of Th2 responses in allergy, and IL-4 secretion after helminth infection (Mack et al., 2005; Min et al., 2004; Sokol et al., 2009; Sullivan and Locksley, 2009; Tschopp et al., 2006; Tsujimura et al., 2008).

Active modulation of macrophage polarization is therefore an approach in the development for anti-inflammatory and anti-cancer therapies.

Additional related background art:

WO2016185026

EP3072525A1

WO02017097876

Wynn TA, Nat Rev Immunol. 2015 May;15(5):271-82.

### SUMMARY OF THE INVENTION

According to an aspect of some embodiments of the present invention there is provided a method of treating a disease or disorder that can benefit from increasing an M2/M1 macrophage ratio in a subject in need thereof, the method comprising:

(a) culturing basophils in the presence of IL33 and/or GM-SCF; and

(b) administering to the subject a therapeutically effective amount of the basophils following the culturing,

thereby treating the disease or disorder that can benefit from increasing an M2/M1 macrophage ratio in the subject.

According to an aspect of some embodiments of the present invention there is provided a therapeutically effective amount of basophils having been generated by culturing in the presence of IL33 and/or GM-SCF for use in treating a disease or disorder that can benefit from increasing an M2/M1 macrophage ratio in a subject in need thereof.

According to some embodiments of the invention, the basophils are blood circulating basophils or derived from the bone-marrow.

According to some embodiments of the invention, the method further comprises prior to (a):

(i) isolating the basophils from bone marrow or peripheral blood;

(ii) differentiating the basophils from the bone marrow or peripheral blood in the presence of IL-3 to as to obtain a differentiated culture;

(iii) isolating from the differentiated culture a cKIT- population.

According to some embodiments of the invention, the (ii) is performed for 8-10 days in culture.

According to some embodiments of the invention, the (a) is performed for up to 48 hours.

According to some embodiments of the invention, the culturing is performed so as to achieve a lung basophil phenotype.

According to some embodiments of the invention, the lung basophil phenotype comprises expression of growth factors and cytokines selected from the group consisting of Csf1, Il6, Il13, L1cam, Il4, Ccl3, Ccl4, Ccl6, Ccl9 and Hgf, the expression being higher than in blood circulating basophils.

According to some embodiments of the invention, the lung basophil phenotype comprises an expression signature of Il6, Il13, Cxcl2, Tnf, Osm and Ccl4.

According to some embodiments of the invention, the lung basophil phenotype comprises an expression signature of Fcera1⁺, Il13ra⁺ (Cd123), Itga2⁺ (Cd49b), Cd69⁺, Cd244⁺ (2B4), Itgam⁺ (Cd11b), Cd63⁺, Cd24a³⁰, Cd200r3⁺, Il2re, Il18rap⁺ and C3ar1⁺.

According to some embodiments of the invention, the basophils are human.

According to some embodiments of the invention, the basophils comprise an expression signature of Fcer1, Il13ra1, Itga2, Cd69, Cd244, Itgam, Cd63, Cd24, Il2ra, Il18rap and C3ar1.

According to some embodiments of the invention, the basophils are autologous to the subject.

According to an aspect of some embodiments of the present invention there is provided a method of treating a disease or disorder that can benefit from increasing an M2/M1 macrophage ratio in a subject in need thereof, the method comprising administering to the subject a therapeutically effective amount of a signaling molecule selected from the group consisting of IL6, IL13 and HGF, thereby treating the disease or disorder that can benefit from increasing an M2/M1 macrophage ratio in the subject.

According to an aspect of some embodiments of the present invention there is provided a therapeutically effective amount of a signaling molecule selected from the group consisting of IL6, IL13 and HGF for use in treating a disease or disorder that can benefit from increasing an M2/M1 macrophage ratio in a subject

According to some embodiments of the invention, the therapeutically effective amount increases the M1/M2 macrophage ratio.

According to some embodiments of the invention, the subject is a human subject.

According to some embodiments of the invention, the administering is in a local route of administration.

According to some embodiments of the invention, the administering is to the lung.

According to some embodiments of the invention, the disease or disorder that can benefit from increasing an M2/M1 macrophage ratio is an inflammatory disease.

According to some embodiments of the invention, the inflammatory disease is selected from the group consisting of: sepsis, septicemia, pneumonia, septic shock, systemic inflammatory response syndrome (SIRS), Acute Respiratory Distress Syndrome (ARDS), acute lung injury, aspiration pneumanitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, cryoporin associated periodic syndromes and cirrhosis.

According to some embodiments of the invention, the disease or disorder that can benefit from increasing an M2/M1 macrophage ratio is an autoimmune disease.

According to some embodiments of the invention, the autoimmune disease is selected from the group consisting of Addison's Disease, Allergy, Alopecia Areata, Alzheimer's disease,

Antineutrophil cytoplasmic antibodies (ANCA)-associated vasculitis, Ankylosing Spondylitis, Antiphospholipid Syndrome (Hughes Syndrome), arthritis, Asthma, Atherosclerosis, Atherosclerotic plaque, autoimmune disease (e.g., lupus, RA, MS, Graves' disease, etc.), Autoimmune Hemolytic Anemia, Autoimmune Hepatitis, Autoimmune inner ear disease, Autoimmune Lymphoproliferative syndrome, Autoimmune Myocarditis, Autoimmune Oophoritis, Autoimmune Orchitis, Azoospermia, Behcet's Disease, Berger's Disease, Bullous Pemphigoid, Cardiomyopathy, Cardiovascular disease, Celiac Sprue/Coeliac disease, Chronic Fatigue Immune Dysfunction Syndrome (CFIDS), Chronic idiopathic polyneuritis, Chronic Inflammatory Demyelinating, Polyradicalneuropathy (CIPD), Chronic relapsing polyneuropathy (Guillain-Barre syndrome), Churg-Strauss Syndrome (CSS), Cicatricial Pemphigoid, Cold Agglutinin Disease (CAD), chronic obstructive pulmonary disease (COPD), CREST syndrome, Crohn's disease, Dermatitis, Herpetiformus, Dermatomyositis, diabetes, Discoid Lupus, Eczema, Epidermolysis bullosa acquisita, Essential Mixed Cryoglobulinemia, Evan's Syndrome, Exopthalmos, Fibromyalgia, Goodpasture's Syndrome, Hashimoto's Thyroiditis, Idiopathic Pulmonary Fibrosis, Idiopathic Thrombocytopenia Purpura (ITP), IgA Nephropathy, immunoproliferative disease or disorder (e.g., psoriasis), Inflammatory bowel disease (IBD), including Crohn's disease and ulcerative colitis, Insulin Dependent Diabetes Mellitus (IDDM), Interstitial lung disease, juvenile diabetes, Juvenile Arthritis, juvenile idiopathic arthritis (JIA), Kawasaki's Disease, Lambert-Eaton Myasthenic Syndrome, Lichen Planus, lupus, Lupus Nephritis, Lymphoscytic Lypophisitis, Meniere's Disease, Miller Fish Syndrome/acute disseminated encephalomyeloradiculopathy, Mixed Connective Tissue Disease, Multiple Sclerosis (MS), muscular rheumatism, Myalgic encephalomyelitis (ME), Myasthenia Gravis, Ocular Inflammation, Pemphigus Foliaceus, Pemphigus Vulgaris, Pernicious Anaemia, Polyarteritis Nodosa, Polychondritis, Polyglandular Syndromes (Whitaker's syndrome), Polymyalgia Rheumatica, Polymyositis, Primary Agammaglobulinemia, Primary Biliary Cirrhosis/ Autoimmune cholangiopathy, Psoriasis, Psoriatic arthritis, Raynaud's Phenomenon, Reiter's Syndrome/Reactive arthritis, Restenosis, Rheumatic Fever, rheumatic disease, Rheumatoid Arthritis, Sarcoidosis, Schmidt's syndrome, Scleroderma, Sjorgen's Syndrome, Stiff-Man Syndrome, Systemic Lupus Erythematosus (SLE), systemic scleroderma, Takayasu Arteritis, Temporal Arteritis/Giant Cell Arteritis, Thyroiditis, Type 1 diabetes, Type 2 diabetes, Ulcerative colitis, Uveitis, Vasculitis, Vitiligo, and Wegener's Granulomatosis.

According to some embodiments of the invention, the disease or disorder that can benefit from increasing an M2/M1 macrophage ratio is a pulmonary disease.

According to some embodiments of the invention, the M2/M1 macrophage comprises alveolar macrophages.

According to some embodiments of the invention the disease or disorder that can benefit from increasing an M2/M1 macrophage ratio is a chronic obstructive pulmonary disease (COPD).

According to an aspect of some embodiments of the present invention there is provided a method of treating a disease or disorder that can benefit from increasing an M1/M2 macrophage ratio in a subject in need thereof, wherein the disorder is not associated with basophilia, the method comprising depleting basophils or activity of the basophils in the subject, thereby treating the disease or disorder that can benefit from increasing an M 1/M2 macrophage ratio in the subject.

According to some embodiments of the invention, the depleting is by an agent which depletes the basopohils or the activity of the basophils.

According to an aspect of some embodiments of the present invention there is provided an agent which depletes basopohils or activity of the basophils for use in treating a disease or disorder that can benefit from increasing an M 1/M2 macrophage ratio in a subject in need thereof.

According to some embodiments of the invention, the agent is directed to at least one basophil marker.

According to some embodiments of the invention, the agent targets FceR 1 a, IL33R and/or CSF2Rb.

According to some embodiments of the invention, the agent targets GM-CSF and/or IL33.

According to some embodiments of the invention, the depleting is effected ex-vivo.

According to some embodiments of the invention, the depleting is effected in-vitro.

According to some embodiments of the invention, the basophils are blood circulating basophils.

According to some embodiments of the invention, the basophils are lung resident basophils.

According to some embodiments of the invention, the depleting is effected in a local manner.

According to some embodiments of the invention, the disease or disorder that can benefit from increasing an M1/M2 macrophage ratio is cancer.

According to some embodiments of the invention, the disease or disorder that can benefit from increasing an M1/M2 macrophage ratio is melanoma.

According to some embodiments of the invention, the disease or disorder that can benefit from increasing an M1/M2 macrophage ratio is pulmonary fibrosis.

According to some embodiments of the invention, aid disease or disorder that can benefit from increasing an M 1/M2 macrophage ratio is selected from the group consisting of cancer, fibrotic diseases.

According to an aspect of some embodiments of the present invention there is provided a method of increasing an M1/M2 macrophage ratio, the method comprising depleting basophils having a lung basophil phenotype from a vicinity of macrophages or depleting activity of the basophils, thereby increasing M1/M2 macrophage ratio.

According to an aspect of some embodiments of the present invention there is provided a method of increasing an M2/M1 macrophage ratio, the method comprising enriching for basophils having a lung basophil phenotype in a vicinity of macrophages or an effector of the basophils, thereby increasing M2/M1 macrophage ratio.

According to some embodiments of the invention, the enriching is by GM-CSF and/or IL33.

According to some embodiments of the invention, the effector is selected from the group consisting of IL6, IL13 and HGF.

According to some embodiments of the invention, the method is effected ex-vivo.

According to some embodiments of the invention, the method is effected in-vivo.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C show a single cell map of lung cells during development. FIG. 1A. Experimental design. Single cells were collected from various time points along lung development. FIG. 1B. Single cell RNA-seq data from immune and non-immune compartments were analyzed and clustered by the MetaCell package (not shown), resulting in a two-dimensional projection of single cells onto a graph representation. 20,931 single cells from 17 mice from all time points were analyzed. 260 meta-cells were associated with 22 cell types and states, annotated and marked by color code. FIG. 1C. Expression quantiles of key cell type specific marker genes on top of the 2D map of lung development. Bars depict UMI distribution of marker genes across all cell types, down-sampled for equal cell numbers.

FIGS. 2A-G show dynamic changes in cellular composition and gene expression during lung development. FIG. 2A. Projection of cells from different time points on the 2D map. FIGS. 2B-C. Cell type distribution of the immune (CD45⁺) (B) and non-immune (CD45⁻) (C) compartments across time points. Time points in A-C are pooled over several correlated biological replicates at close time intervals (not shown). FIG. 2D. Dynamic changes in macrophage compartment composition plotted before and after birth (hours; t₀=birth). Dots represented biological samples (n=15). Trend line is computed by local regression (Loess). FIG. 2E. Suggested trajectory from monocytes to macrophage II-III on the 2D map. FIG. 2F. Gene expression profiles of monocytes and macrophage II-III cells ordered according to Slingshot pseudo-time trajectory (Methods). Lower color bars indicate annotation by cell type (middle) and time point of origin (bottom). FIG. 2G. Expression of hallmark monocyte and macrophage genes across meta-cells. Meta-cells are ordered by median pseudo-time; five left-most meta-cells are macrophage I.

FIGS. 3A-I show lung resident basophils broadly interact with the immune and other compartments. FIG. 3A. Illustration of ligand receptor map analysis. Each node is a ligand or receptor, and a line represents an interaction. FIG. 3B. The ligand-receptor map of lung development pooled across all time-points. Genes (ligands and receptors) were projected on a 2D map based on their correlation structure (Methods). Genes related to specific cells were marked by their unique colors, according to FIGS. 1A-C. FIG. 3C. Projection of genes activated in the immune (green) and non-immune (red) compartments. Full and empty circles represent ligands and receptors, respectively. Gray circles represent ligand/receptors non-specific to one compartment. FIGS. 3D-E. Ligands were classified to functional groups by GO-enrichment (Methods). FIG. 3D. Enrichment of functional groups of ligands in the immune and non-immune compartments. FIG. 3E. Enrichment of receptors whose ligands are from different functional groups in the immune and non-immune compartments. FDR corrected Fisher exact test; p<0.05. FIG. 3F-I. LR interaction maps of smooth-muscle fibroblasts (F), AT2 cells (G),

ILC (H) and basophils (I). Colored nodes represent genes up-regulated in the cell type (>2 fold change), and gray nodes represent their interacting partners. Full and empty circles represent ligands and receptors, respectively. *p<0.05, **p<0.01, ***p<0.005.

FIGS. 4A-G show spatial and transcriptomic characterization of lung basophils. FIG. 4A. Detection of alveoli, nuclei and basophils in whole lobe sections of Mcpt8^{YFP/+} mice by TissueFAXS. Inlet: red arrows point at YFP⁺ basophils. Bottom: output of computational analysis showing alveoli (white), nuclei (gray) and basophils (yellow). Heat colors indicate distance from nearest alveoli (Methods). Scale bar=lmm (whole lobe) and 20 μm (representative section) FIG. 4B. Quantification of basophil (yellow) distance from the alveoli compared to all other nuclei (gray) at day 8.5 PN and 8 weeks adult mice. Distances were normalized to median value across all nuclei. P-values calculated by permutation test (Methods). n=4-5 mice per group. FIG. 4C. Representative images of Mcpt8⁺ basophils (green) in cleared lungs derived from 30h PN, day 6.5 PN and 8 weeks adult mice. Scale bar=2mm. FIG. 4D. Quantification of lung basophil numbers in whole lungs at different developmental time points by flow cytometry. n=3-4 mice per group. One-way ANOVA; Student's t-test (two tailed) between 8w and day 6.5 PN and between 8w and 30h PN. FIG. 4E. Differential gene expression of basophils derived from lung (y axis) and peripheral blood (x axis) at 30h PN. FIG. 4F. Expression of ligands specific to lung basophils across blood and lung basophils at E16.5, 30h PN and 8 weeks. Values for FIGS. 4E-F indicate normalized expression per 1,000 UMI scaled to number of cells. FIG. 4G. Distribution of lung basophil specific signature (FIG. 7G) across blood and lung basophils from time-matched developmental time-points. Box plots display median bar, first-third quantile box and 5th-95th percentile whiskers. *p<0.05, **p<0.01.

FIGS. 5A-L show lung resident basophils are primed by IL33 and GM-CSF. FIG. 5A. Dual projection of the ligand Csf2 (green) and its unique receptor Csf2rb (red) on the single cell map from FIGS. 1A-C. Colors indicate expression quantiles. Bar plots indicate ligand and receptor normalized expression per 1,000 UMI across cell types. FIG. 5B. Quantification of CSF2Rb⁺ lung basophils compared to mast cells and total CD45⁺ cells at 30h PN by flow cytometry; n=2 mice per group. One-way ANOVA: Student's t-test (two tailed) between basophils and mast cells. FIG. 5C. As FIG. 5A but for the ligand 1133 (green) and its unique receptor Il1rl1(red). FIG. 5D. As FIG. 5B but for IL1RL1⁺ lung basophils; n=3 mice per group. FIG. 5E. Representative smFISH image of mRNA molecules for Mcpt8 (red), a marker for basophils, Il33 (green), a ligand expressed by AT2 cells, and Il1rl1 (white), the counterpart receptor expressed by basophils, together with DAPI staining (blue) to mark cell nuclei, in lung tissue derived from 8 days PN; Scale bar=5 μm. FIG. 5F. Representative IHC image of Mcpt8³⁰ basophils (brown) and pro-SPC⁺ AT2 cells (purple), together with methylgreen staining for cell nuclei detection (green), in a lung section derived from adult (8 weeks) mice, showing their spatial proximity to each other and to the alveoli. Scale bar=25 μm FIG. 5G. Differential gene expression between 30h PN lung basophils from Il1rl1 (ST2) knockout (y axis) versus littermate controls (x axis). Values indicate loge normalized expression per 1,000 UMI /cells. FIG. 5H. Distribution of lung basophil specific signature (FIG. 7G) in Il1rl1 knockout and littermate controls. Box plots display median bar, first-third quantile box and 5th-95th percentile whiskers. FIG. 5I. Illustration of experimental paradigm for in vitro culture. BM-derived cells were grown with IL3 to induce basophils for 10 days and then cKit⁻ cells were sorted for plating (FIG. 5J). Basophils were plated for 16h with IL3 alone (a), IL3 and GM-CSF (b) IL3 and IL33 (c) and a combination of IL3, IL33 and GM-CSF (d). Gene expression of single cell sorted basophils was evaluated by MARS-seq. FIG. 5J. Expression of key genes across the four conditions. Values indicate normalized expression per 1,000 UMI /cells. FIG. 5K. Scoring meta-cells from the four conditions for their expression of the IL33 induced program (y axis) and the GM-CSF induced program (x axis; FIG. 5L). Meta-cell identity is determined by the majority of cells. FIG. 5L. Scoring meta-cells from 30h PN lung (filled red circles) and blood circulating (empty red circles) basophils, and adult (8 weeks) lung (filled brown circles) and blood circulating (empty brown circles) basophils projected on the gene-expression programs described in FIG. 5K. FIGS. 5J-L. Samples were prepared in triplicates, and results are representative of three independent experiments. *p<0.05, **p<0.01. Data are represented as mean±SEM.

FIGS. 6A-P Lung basophils are essential for transcriptional and functional development of AM. FIG. 6A. Dual projection of the ligand Il16 (green) and its unique receptor Il6ra (red) on the single cell map from FIGS. 1A-C. Colors indicate expression quantiles. Bar plots indicate ligand and receptor normalized expression per 1,000 UMI across cell types. FIG. 6B. Histogram and quantification of intracellular staining of IL-6, compared to isotype control, within lung basophils, mast cells and total CD45⁺ cells at 30h PN, by flow cytometry; n=6 mice per group. FIG. 6C. As in FIG. 6A but for Il13 (green) and its receptor Il13ra1 (red). FIG. 6D. As in FIG. 6B but for IL-13; n=6 mice per group; FIGS. 6A-D. One-way ANOVA; Student's t-test (two tailed) between basophil and mast cells. FIG. 6E. Representative IHC image of Mcpt8⁺ basophils (dark purple) and F4/80⁺ macrophages (brown), on hematoxylin staining (light purple), in lung section derived from 8 days PN mice, showing their spatial proximity; Scale bar=40 μm. FIGS. 6F-I. Newborn mice were injected intra-nasally with anti-Fcεr1α antibody for basophils depletion or with isotype control, and viable CD45⁺ cells were sorted for MARS-seq processing and analysis at 30h PN. Each sample was pooled from three lungs, and results are representative of three replicates in two independent experiments. FIG. 6F. Fraction of basophils (Fcεr1α⁺cKit⁻) from total CD45⁺ cells in lungs derived from anti-Fcεr1α and isotype control injected mice, as determined by FACS. Student's t-test (two tailed) for percent of lung basophils; n=3. FIG. 6G. Fraction of Macrophage III from total macrophages in lungs derived from anti-Fcεr1α and isotype control injected mice. Numbers were scaled to match control levels between experiments. Student's t-test (two tailed) for percent of AM. FIG. 6H. Expression of genes differentially expressed between Macrophage II (light green) and macrophage III (dark green) cells in anti-Fcεr1α (y axis) and isotype control (x axis) treated mice. Values indicate normalized expression per 1,000 UMI /cells. FIG. 6I. Median expression of hallmark AM and Macrophage II (F13a1) genes in anti-Fcεr1α versus isotype control treated mice. FIGS. 6J-K. AM derived from BALF of Mcpt8 knockout and their littermate controls were purified from adult, 8-12 weeks old mice. Results are from four independent experiments; each of them consists of at least four replicates. FIG. 6J. BALF cell count of Mcpt8 knockout and their littermate control mice. Student's t-test for percent of AM. FIG. 6K. Phagocytosis capacity of AM derived from BALF of Mcpt8 knockout versus littermate control mice. Results are shown as fold change of phagocytosis index compared to averaged controls. Student's t-test for percent of AM. FIGS. 6L-P. Co-culture experiment of BM-MΦ and BM-derived basophils. BM derived cells were split and grown into basophils (IL3) for 10 days, and macrophages (M-CSF) for 8 days. Macrophages were then co-cultured with (a) M-CSF+IL3, (b) IL33 and GM-CSF, (c) naive basophils and (d) lung milieu-primed basophils in the presence of IL33 and GM-CSF. FIG. 6L. A two-dimensional representation of the meta-cell analysis of co-cultured macrophages from the four conditions. Right-Expression quantile of selected AM related genes onto the 2D projection. FIG. 6M. A lung milieu-primed basophil induced program in co-cultured macrophages is associated with macrophage priming toward AM and immune suppression. Biological replicates are shown. FIG. 6N. Differential expression (log2 fold change) of the genes in M between Macrophage III and II during development. FIG. 6O. Expression of the genes in M across CD45⁺CD115⁺ myeloid cells sorted from 30h PN lungs, grown under the same conditions as in FIG. 6M. Biological replicates are shown. FIG. 6P. Differential expression (log2 fold change) of the genes in M between macrophages derived from lungs injected with anti-Fcεr1α and isotype control. *p<0.05, **p<0.01, ***p<0.001. Data are represented as mean±SEM.

FIGS. 7A-I provide additional data related to spatial and transcriptomic characterization of lung basophils FIG. 7A. Representative IHC images of Mcpt8⁺ basophils (brown; red arrows) with hematoxylin background in lung section derived from E16.5, 30h PN, day 8.5 PN and 8 week adult mice n=3-5 for each time point. FIG. 7B. Lung cells derived from day 2 PN mice were enriched for basophils, by single cell sorting according to specific cell-surface markers. Protein levels of cKit and Fcεr1α of CD45⁺ cells were determined by FACS index sorting. Cells are colored by association to cell type as in FIGS. 1A-C, by transcriptional similarity (Method). FIG. 7C. Cell type distribution of the cKit⁺, Fcεr1α⁺ and double negative (DN) gates as in FIG. 7B. FIG. 7D. Quantification of YFP⁺ fraction in lung cells derived from Mcpt8^{YFP/+} transgenic neonates at 30h PN, and enriched for basophils (CD45⁺ cKit⁻ Fcεr1α⁺), compared to mast cells (CD45⁺cKit⁺) and the CD45⁺ compartment; n=6. Student's t-test (two tailed): ***p<0.001. FIG. 7E. Quantification of CD49b±lung basophils compared to mast cells and total CD45⁺ cells at 30h PN by flow cytometry; n=6. One-way ANOVA: ***P<0.001; Student's t-test (two tailed) between basophil and mast cells: ***p<0.001; Data are represented as mean±SEM. FIG. 7F. Gating strategy for basophils derived from blood circulation (low panel) and lung parenchyma (upper panel) at E16.5, 30h PN and 8 weeks old mice, according to Fcεr1α⁺cKit⁻ expression. FIG. 7G. Differential gene expression between lung and blood basophils in 30h PN (y axis) and adult (8 weeks, x axis) mice. Inlet displays percentages of differentially expressed genes (fold change >1) in each quartile. Red genes were selected for the definition of the lung basophil signature (FIGS. 4A-G, 5A-L). FIG. 7H. Specificity of basophils expressed ligands across all lung cell types. Expression threshold is 2-fold change (not shown). Colors represent cell types, as in FIGS. 1A-C. FIG. 71. Expression of ligands exclusively expressed by basophils compared to all cell types. ***p<0.001. Data are represented as mean±SEM.

FIGS. 8A-G provide additional data related to lung resident basophils are primed by IL33 and GM-CSF. FIG. 8A. Gene expression similarity of Il1rl1 knockout, or its littermate control, lung basophils to lung or blood basophils derived from mice at 30h PN. Each Il1rl1 KO cell was assigned to either blood or lung by k nearest neighbor majority voting (Methods). FIGS. 8B-E. BM-derived cells were grown with IL3 to induce basophils for 10 days and then cKIT⁻cells were sorted for plating. Basophils were plated for 16h with IL3 alone (a), IL3 and GM-CSF (b) IL3 and IL33 (c) and a combination of IL3, IL33 and GM-CSF (d). FIG. 8B. BM-derived cells were enriched for BM-basophils by negative selection using cKit beads. Percentage of pure BM-basophil population out of total BM cells was evaluated by FACS. FIG. 8C. Heat-map represents gene expression profiles of basophils that were grown with different combinations of the cytokines. Color bar indicates a-d cytokine combinations. FIG. 8D. Differential gene expression between basophils grown with one cytokine (x axis—GM-CSF; y axis—IL33) and naïve basophils (grown with IL3 alone). Horizontal and vertical intercepts indicate thresholds for IL33 and GM-CSF induced gene programs, respectively. FIG. 8E. Distribution of lung basophil specific signature (FIG. 7G) in BM-derived basophils grown under the four conditions. Box plots display median bar, first-third quantile box and 5th-95th percentile whiskers. **P=0.009; Kolmogorov-Smirnov test. FIG. 8F. Scoring biological replicates from the a-d cytokine conditions for their expression of the IL33 induced program (y axis) and the GM-CSF induced program (x axis). Conditions a and d are from three independent experiments. FIG. 8G. Scoring meta-cells from the Il1rl1 knockout lung basophils and their littermate controls at 30h PN, for their expression of the IL33 induced program (y axis) and the GM-CSF induced program (x axis).

FIGS. 9A-N provide additional data related to lung basophils are essential for transcriptional and functional development of AM. FIG. 9A. Dual projection of the ligand Csf1 (green) and its unique receptor Csf1r (red) on the single cell map from FIGS. 1A-C. Colors indicate expression quantiles. Bar plots indicate ligand and receptor normalized expression per 1,000 UMI across cell types. FIG. 9B. Illustration of the basophil depletion experiment. Newborn mice were injected intra-nasally with anti-Fcεr1α antibody for basophils depletion or with isotype control twice, at 12h and 16h PN, and viable CD45⁺ cells were sorted for MARS-seq processing and analysis at 30h PN. FIG. 9C. Gating strategy for CD45⁺Fcεr1α⁺cKit⁻ lung basophils derived from anti-Fcεr1α or isotype control injected neonates. FIG. 9D. Frequency of different cell types from total CD45⁺ cells in lungs derived from anti-Fcεr1α and isotype control injected mice, as determined by mapping single cells to the lung model (FIGS. 1A-C, Methods). Numbers were scaled to match control levels between experiments. Student's t-test (two tailed): *p=0.02; n=3. FIG. 9E. Expression difference of the most differentially expressed genes between macrophages subsets II (light-green) and III (dark-green), when comparing lung macrophages derived from anti-Fccrla and isotype control injected mice. Shown are the top 15 differentially expressed genes on both sides. Values represent log2 fold change. FIG. 9F. Distribution of macrophage III specific gene expression across macrophages derived from anti-Fcεr1α and isotype control injected mice. Expression level was scaled to match control levels between experiments. Kolmogorov-Smirnov test; ***p<10⁻⁴. FIG. 9G. Percentage of AM out of CD45⁺ cells derived from BALF of Mcpt8 knockout and their littermate controls at adult, 8-12 weeks old mice. FIG. 9H. BM derived cells were split and grown into basophils (IL3) for 10 days, and macrophages (M-CSF) for 8 days. Macrophages were then co-cultured with (a) M-CSF+IL3, (b) IL33 and GM-CSF, (c) BM-derived basophils and (d) lung milieu-primed basophils (in the presence of IL33 and GM-CSF). FIG. 91. Differential gene expression between basophils grown with GM-CSF and IL33 and naive basophils. Basophils were grown alone (x axis), or in the presence of macrophages (y axis). Inlet displays fraction of differentially expressed genes (fold change >1) in each quartile. FIG. 9J. Heat-map represents gene expression profiles of BM-MΦ grown with and without basophils as in FIG. 6L. Color bar indicates a-d growth conditions. FIG. 9K. Differential gene expression between macrophages grown with or without lung basophils (conditions a and d). Axes represent two independent experiments. Inlet displays fraction of differentially expressed genes (fold change >1) in each quartile. FIG. 9L. Distribution of the immune-modulating specific gene expression induced by lung resident basophils across Macrophage II and III in lung development. Kolmogorov-Smirnov test; ***p<10⁻¹⁰. FIGS. 9M-N. Comparison of basophil gene expression derived from different tissues. FIG. 9M. Gene expression of basophil hallmark genes (Mcpt8, Cpa3, Cd200r3), as well as tissue specific genes (Il6, Ccl3), across basophils collected from lung, tumor microenvironment, blood, spleen and liver of 8 weeks old mice. Non-basophils indicate cells collected and filtered as outliers. FIG. 9N. Distribution of gene expression signature of the lung basophils (FIG. 7G) across basophils derived from different tissues. *p<0.05, ***p<0.001.

### DESCRIPTION OF SPECIFIC EMBODIMENTS OF THE INVENTION

The present invention, in some embodiments thereof, relates to methods of modulating M2 macrophage polarization and use of same in therapy.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Macrophages derived from monocyte precursors undergo specific differentiation depending on the local tissue environment. The various macrophage functions are linked to the type of receptor interaction on the macrophage and the presence of cytokines. Similar to the T helper type 1 and T helper type 2 (TH1-TH2) polarization, two distinct states of polarized activation for macrophages have been defined: the classically activated (M1) macrophage phenotype and the alternatively activated (M2) macrophage phenotype. Similar to T cells, there are some activating macrophages and some suppressive macrophages, therefore, macrophages should be defined based on their specific functional activities. Classically activated (M1) macrophages have the role of effector cells in TH1 cellular immune responses. The alternatively activated (M2) macrophages appear to be involved in immunosuppression and tissue repair. For these reasons, modulating the ratio of Ml/M2 has been considered as a relevant approach for the treatment of inflammation and autoimmunity on the one hand and cancer on the other hand.

Whilst reducing the present invention to practice, the present inventors have identified a lung-resident population of basophils that reside in close proximity to alveoli. These basophils are characterized by a unique gene expression phenotype and cytokine/growth factor secretion. They play an important role in guiding the maturation and function of alveolar macrophages in the lung. It is suggested that a lung resident basophil phenotype is also a hallmark of disease conditions which are not limited to the lung, suggesting that they can be beneficial towards treating medical conditions that can benefit from Ml/M2 modulation.

Specifically, the present inventors report the extensive profiling of immune and non-immune lung cells by single cell RNA-sequencing of 50,770 cells along major time points of lung development. A highly diverse set of cell types and states was observed, and complex dynamics of developmental trajectories were identified, including three waves of macrophage types, from primitive cells to mature AM. Analysis of interacting ligands and receptors revealed a highly connected network of interactions, and highlighted basophils as cells expressing major growth factors and cytokine signaling in the lung. Basophils in the lung reside in close proximity to alveoli, and exhibit a lung specific phenotype, highly diverged from peripheral circulating basophils. Using Il1rl1 (IL-33 receptor) knockout mice and in vitro cultures, the present inventors discovered that lung basophils' education is mediated by the combinatorial imprinting of GM-CSF (Csf2) and IL-33 from the lung environment, and can be recapitulated in vitro by introducing these cytokines. Using antibody depletion strategies, diphtheria toxin-mediated selective depletion of basophils and in-vitro co-culture experiments, the present inventors demonstrate that basophils play an important role in guiding the maturation and function of alveolar macrophages (AM) in the lung. These findings open new clinical strategies to macrophage manipulation and basophil-based therapeutics.

Thus, according to an aspect of the invention, there is provided a method of increasing an M2/M1 macrophage ratio. The method comprises enriching for basophils having a lung basophil phenotype in a vicinity of macrophages or an effector of said basophils, thereby increasing M2/M1 macrophage ratio.

As used herein “M1 macrophages” refer to macrophages characterized by the expression of proinflammatory genes and are typically endowed with an effector function in TH1 cellular immune responses. M1 macrophages according to some embodiments of the present invention can be identified by using FACS, or by their cytokine secretion profile (e.g., TNFa, IL1b), and can be quantified by ELISA for instance or at the RNA level such as by using RT-PCR.

As used herein “M2 macrophages” refer to macrophages that are endowed with an immunosuppression activity and tissue repair. M2 macrophages according to some embodiments of the present invention can be quantified by cell number using specific markers (e.g., MRC1, ARG1) such as by using FACS, or by their cytokine secretion profile (e.g., IL-10, CCL17, CCL22) and can be quantified by ELISA for instance or at the RNA level such as by using RT-PCR.

As used herein “alveolar macrophages” or “AM” refer to a type of macrophages found in the pulmonary alveolus. AM originate from fetal liver embryonic precursors and are self-maintaining, with no contribution from the adult bone marrow.

Mouse AM can be identified using anti-CD45, anti-CD11c, anti-F4/80 and/or anti-SIGLEC-F.

Human AM can be identified using anti-CD45 and/or anti-CD11c

As used herein “increasing” refers to at least 10%, 20%, 30%, 40%, 50%, 60%, 70%, 80%, 85%, 90% or even 95%, increase in M2/M1 ratio (M2 polarization) as compared to that in the absence of said enrichment (e.g., GM-CSF, IL33, IL6 and/or IL13), as assayed by methods which are well known in the art (see Examples section which follows).

Increasing an M2/M1 macrophage ratio refers to M2 polarization.

As mentioned, the method of this aspect of the invention is performed by enriching for basophils having a lung basophil phenotype

The present inventors have shown that a lung basophil phenotype can be acquired in vitro (see Examples section which follows).

As used herein “a lung basophil phenotype” refers to a structural and/or functional phenotype.

According to a specific embodiment, the structural phenotype comprises a signature of Fcera1⁺, Il3ra⁺ (Cd123), Itga2⁺ (Cd49b), Cd69⁺, Cd244⁺ (2B4), Itgam⁺ (Cdl11b), Cd63⁺, Cd24a⁺, Cd200r3⁺, Il2ra⁺, Il18rap⁺ and C3ar1⁺; or Fcer1⁺, Il13ra1⁺, Itga2⁺, Cd69⁺, Cd244⁺, Itgam⁺, Cd63⁺, Cd24⁺, Il2ra⁺, Il18rap⁺ and C3ar1+ in the case of human cells.

According to an additional or alternative embodiment, the structural phenotype comprises expression of key cytokines and growth factors, such as Csf1, Il6, Il13, L1 cam, Il4, Ccl3, Ccl4, Ccl6, Ccl9 and Hgf.

According to an additional or alternative embodiment, the structural phenotype comprises expression of key cytokines and growth factors Il6, Il13, and Hgf.

According to an additional or alternative embodiment, the structural phenotype comprises a distinct gene expression profile of lung basophils from blood-circulating basophils, characterized by a unique gene signature that includes expression of Il6, Il13, Cxcl2, Tnf, Osm and Ccl4

A “functional phenotype” refers to the effect of M2 polarization on macrophages.

According to a specific embodiment, the basophils are mammalian basophils.

According to a specific embodiment, the basophils are human basophils.

According to an embodiment, the enriching is by contacting with GM-CSF and/or IL33.

According to an embodiment, the enriching is by contacting with GM-CSF and IL33.

As used herein “contacting” or methods described herein can be performed, in-vivo, ex-vivo or in-vitro.

According to a specific embodiment, the enriching is effected in vitro or ex vivo.

As used herein “basophils” refer to a specific type of leukocytes called granulocytes, which are characterized by large cytoplasmic granules that can be stained by basic dyes and a bi-lobed nucleus, being similar in appearance to mast cells, another type of granulocyte. Basophils are the least common granulocyte, making only 0.5% of the circulating blood leukocytes, and have a short life span of only 2-3 days (in vivo). Basophils are derived from granulocyte-monocyte progenitors in the bone marrow; where basophil precursors and mast cell precursors arise from an intermediate bipotent basophil-mast cell precursor (Arinobu et al. 2005 and Arinobu et al. 2009). Table 1 shows the markers associated with the different lineage cell types.

TABLE 1

| Cell Type | Markers |
|---|---|
| Granulocyte-monocyte progenitors | IL-7Rα⁻, Lin⁻, Sca-1⁻, c-Kit⁺, |
| | CD34⁺, FcγRII/III^{hi}, β7^{lo} |
| Intermediate bipotent basophil-mast | Lin⁻, c-Kit⁺, FcεRII/HI^{hi}, β7^{hi} |
| cell precursor | |
| Basophil precursor | c-Kit⁻, FcεRI⁺, CD11b⁺ |
| Mast cell precursor | c-Kit^{hi}, FcεRI⁺, CD11b⁻ |

Data from Min et al 2012 Immunol. 135, 192-197.

Basophils can be identified by the expression of certain markers, which is consistent between humans and mice, refer to Table 2.

TABLE 2

| Human and Mice Markers - | Human and Mice Markers - |
|---|---|
| Present/Positive | Absent/Negative |
| FcεRI^{hi} | B220 |
| IgE^{hi} | CD3 |
| CD49b^{hi} | CD23 |
| IL-3R^{hi} | CD117 |
| CD13 (up regulated when activated) | Gr-1 |
| CD24 | Ly-49c |
| CD33 | NK1.1 |
| CD43 | αβTCR |
| CD44 | γΥTCR |
| CD45 | |
| CD54 | |
| CD63 | |
| CD69 | |
| CD107a (up regulated when activated) | |
| CD123 | |
| CD164 (up regulated when activated) | |
| CD193 | |
| CD194 | |
| CD203c | |
| CD294 | |
| Siglec-8 | |
| TLR-4 | |
| Thy-1.2 | |

Data from Schroeder 2009 Ad. Immunol. Adv Immunol. 101, 123-161, Hida et al 2009 Nat. Immunol. 10, 214-222. and Heneberg 2011 Cu. Pharm. Design 17, 3753-3771.

According to a specific embodiment, basophils are isolated from the bone-marrow or peripheral blood.

According to a specific embodiment, basophils are produced as follows:

(i) isolating the basophils from bone-marrow.

(ii) differentiating the basophils from the peripheral blood in the presence of IL-3 to as to obtain a differentiated culture;

(iii) isolating from the differentiated culture a cKIT⁻population.

According to an exemplary protocol, bone marrow (BM) progenitors are harvested and cultured at a predetermined concentration e.g., of 0.1×10⁶-1×10⁶ cells per ml. For BM-derived macrophages (MΦ) differentiation, BM cells are cultured for 6-10 days, e.g., 8 days, in the presence of M-CSF. Then, cells are scraped. For BM-derived basophils differentiation, BM cells are cultured for 7-10 days, in the presence of IL-3 (e.g., 9-10 days). Following, basophils are enriched by magnetic-activated cell sorting for a CD117⁻population (cKit; Miltenyi Biotec), and re-plated for 16 hours. During differentiation, cultures can be in standard media.

Ex-vivo methods can be done in tissue culture or when possible in a closed system such as by apheresis.

Bone marrow cultures or circulating basophils (peripheral blood) cultures are treated with the differentiation factors. Culturing can be effected while supplementing with IL-3 (5-20 ng/ml, e.g., 10 ng/ml) and M-CSF (5-20 ng/ml, e.g., 10 ng/ml) for cell survival; and/or IL33 (30-70 ng/ml, e.g., 50 ng/ml) and/or GM-CSF (30-70 ng/ml, e.g., 50 ng/ml) for cell activation towards basophils that can regulate M2 polarization of macrophages. Typically, cell activation is performed for 48 hours or less, e.g., 6-48 hours, 12-48 hours, 24-48 hours, 12-36 hours, 18-24 hours, e.g., 24 hours (e.g., IL33+GM-CSF).

As used herein “in a vicinity of macrophages” can refer to a co-culture of basophils and macrophages. Alternatively, “in a vicinity of macrophages” can refer to enriching such that there is an effective amount of basophils having a lung basophil phenotype in vivo, or an effective amount of effectors of said basophils so as to allow polarization to M2 macrophages.

Effectors of basophils having a lung basophil phenotype include, but are not limited to IL6, IL13 and/or HGF (hepatocyte growth factor).

According to another aspect there is provided, a method of increasing an M 1/M2 macrophage ratio, the method comprising depleting basophils having a lung basophil phenotype from a vicinity of macrophages or depleting activity of said basophils, thereby increasing M1/M2 macrophage ratio.

Increasing M1/M2 macrophage ratio also refer to M1 polarization.

As used herein “increasing” refers to at least 10%, 20%, 30%, 40%, 50%, 60%, 70%, 80%, 85%, 90% or even 95%, increase in M1/M2 ratio (M1 polarization) as compared to that in the absence of said depletion, as assayed by methods which are well known in the art (see Examples section which follows).

Depleting basophils having a lung basophil phenotype can be effected by any method known in the art, some are described infra.

According to an embodiment, depletion can be effected by an agent targeting a basophil marker.

Such markers are described hereinabove e.g., Fcera1⁺, Il3ra⁺ (Cd123), Itaga2⁺ (Cd49b), Cd69⁺, Cd244⁺ (2B4), Itgam⁺ (Cd11b), Cd63⁺, Cd24a⁺, Cd200r3⁺, Il2ra⁺, Il18rap⁺ and C3ar1⁺; or Fcer1⁺, Il13ra1⁺, Itga2⁺, Cd69⁺, Cd244⁺, Itgam⁺, Cd63⁺, Cd24⁺, Il2ra⁺, Il18rap⁺ and C3ar 1⁺or as listed in Table 2.

According to a specific embodiment, the depletion is effected to specifically eliminate basophils having a lung basophil phenotype and not other cell populations (depletion of other cell populations is not affected by more than 20%, 15%, 10%, 5%, 1%, each value is considered a different embodiment).

According to a specific embodiment, such an agent can be an antibody such as an anti Fceral+antibody.

The choice of antibody type will depend on the immune effector function that the antibody is designed to elicit.

According to specific embodiments, the antibody comprises an Fc domain.

According to specific embodiments, the antibody is a naked antibody.

As used herein, the term “naked antibody” refers to an antibody which does not comprise a heterologous effector moiety e.g. therapeutic moiety.

According to specific embodiments, the antibody comprises a heterologous effector moiety typically for killing the basophils thereby increasing M1/M2 macrophage ratio. The effector moiety can be proteinaceous or non-proteinaceous; the latter generally being generated using functional groups on the antibody and on the conjugate partner. The effector moiety may be any molecule, including small molecule chemical compounds and polypeptides. Non-limiting examples of effector moieties include but are not limited to cytokines, cytotoxic antibodies, toxins, radioisotopes, chemotherapeutic antibody, tyrosine kinase inhibitors, and other therapeutically active antibodies. Additional description on heterologous therapeutic moieties is further provided hereinbelow.

The antibody may be mono-specific (capable of recognizing one epitope or protein), bi-specific (capable of binding two epitopes or proteins) or multi-specific (capable of recognizing multiple epitopes or proteins).

According to specific embodiments, the antibody is a mono-specific antibody.

According to specific embodiments, the antibody is bi-specific antibody.

Bi-specific antibodies are antibodies that are capable of specifically recognizing and binding at least two different epitopes. The different epitopes can either be within the same molecule or on different molecules such that the bi-specific antibody can specifically recognize and bind two different epitopes on a single RTN4 polypeptide as well as two different polypeptides. Alternatively, a bi-specific antibody can bind e.g. RTN4 and another effector molecule such as, but not limited to e.g. CD2, CD3, CD28, B7, CD64, CD32, CD16. Methods of producing bi-specific antibodies are known in the art and disclosed for examples in U.S. Pat. Nos. 4,474,893, 5,959,084, and 7,235,641, 7,183,076, U.S. Publication Number 20080219980 and International Publication Numbers WO 2010/115589, WO2013150043 and WO2012118903 all incorporated herein by their entirety; and include, for example, chemical cross-linking (Brennan, et al., Science 229,81 (1985); Raso, et al., J. BioI. Chern. 272, 27623 (1997)), disulfide exchange, production of hybrid-hybridomas (quadromas), by transcription and translation to produce a single polypeptide chain embodying a bi-specific antibody, or by transcription and translation to produce more than one polypeptide chain that can associate covalently to produce a bi-specific antibody. The contemplated bi-specific antibody can also be made entirely by chemical synthesis.

Antibodies with more than two valencies are also contemplated.

According to other specific embodiments, the antibody is a multi-specific antibody.

According to specific embodiments, the antibody is a conjugate antibody (i.e. an antibody composed of two covalently joined antibodies).

The antibody may be monoclonal or polyclonal.

According to specific embodiments, the antibody is a monoclonal antibody.

According to specific embodiments, the antibody is a polyclonal antibody.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

It will be appreciated that for human therapy or diagnostics, humanized antibodies are preferably used.

According to specific embodiments, the antibody is a humanized antibody. Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

According to another embodiment, the depletion is effected by depleting activity of the basophils so as to prevent signal communication with the macrophages.

According to a specific embodiment, such an activity is of IL6, IL13 and/or HGF.

Inhibiting the activity of any of these molecules can be done using antibodies for those ligands, or soluble receptors, also referred to as “decoys” that bind to these ligands and prevent their function.

Typically, such soluble receptors comprise the extracellular portion of the receptor molecule and are devoid of the transmembrane domain(s) and the cytoplasmic domain(s).

The receptor of HGF is c-Met receptor.

The receptor for IL6 is Interleukin 6 receptor (IL6R) also known as CD126.

The receptor for IL13 is interleukin-13 receptor.

Small molecule inhibitors of c-MET, IL6R and IL13R are well known in the art and some are already in clinical use. Examples of c-Met inhibitors include, but are not limited to, class I and class II ATP-competitive small molecule c-Met inhibitors, e.g., JNJ-38877605, PF-04217903, XL880, foretinib and AMG458, as well as ATP-non-competitive small molecule c-Met inhibitors such as, Tivantinib (ARQ197). Examples of IL6R inhibitors (e.g, antibodies, Tocilizumab, Sarilumab), small molecules inhibitors of IL6 are taught in WO2013019690, incorporated hereinby reference. An examples of IL13R inhibitor is ASLAN004.

In order to ensure specificity to a specific tissue (when needed), the agent can be accompanied by a specific delivery vehicle e.g., directed to a tissue marker or administered in a local manner e.g., for pulmonary activity e.g., intranasal administration. Modes of administration are described hereinbelow.

As used herein “depletion” refers to at least 10%, 20%, 30%, 40%, 50%, 60%, 70%, 80%, 90% or more, even total elimination as determined by FACS of the desired cells, be them basophils of a lung phenotype or M2 macrophages.

Methods of Detecting the Expression Level of RNA

The expression level of the RNA in the cells of some embodiments of the invention can be determined using methods known in the arts.

Northern Blot analysis: This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RT-PCR analysis: This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls.

RNA in situ hybridization stain: In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the bound probe is detected using known methods. For example, if a radio-labeled probe is used, then the slide is subjected to a photographic emulsion which reveals signals generated using radio-labeled probes; if the probe was labeled with an enzyme then the enzyme-specific substrate is added for the formation of a colorimetric reaction; if the probe is labeled using a fluorescent label, then the bound probe is revealed using a fluorescent microscope; if the probe is labeled using a tag (e.g., digoxigenin, biotin, and the like) then the bound probe can be detected following interaction with a tag-specific antibody which can be detected using known methods.

In situ RT-PCR stain: This method is described in Nuovo GJ, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, Calif.).

Methods of Detecting Expression and/or Activity of Proteins

Expression and/or activity level of proteins expressed in the cells of the cultures of some embodiments of the invention can be determined using methods known in the arts.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-immunoassay (RIA): In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical analysis: This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

Ex-vivo or in-vitro cells or cell populations obtainable by any of the methods described herein are also contemplated according to some embodiments of the invention. Cell populations obtained according to some embodiments of the invention are characterized by a level of purity higher than that found in the physiological environment (e.g., at least 30%, 40%, 50%, 60%, 70%, 80%, 90% or more of the cells are the cells of interest e.g., basophils, or cells differentiated therefrom or macrophages).

As mentioned any of the methods described can be effected ex-vivo or in-vivo.

The ability to modulate the balance between M1 and M2 macrophages, allows harnessing the present teachings towards therapy.

Thus, according to an aspect of the invention there is provided a method of treating a disease or disorder that can benefit from increasing an M2/M1 macrophage ratio in a subject in need thereof, the method comprising:

(a) culturing basophils in the presence of IL33 and/or GM-SCF; and

(b) administering to the subject a therapeutically effective amount of the basophils following the culturing,

thereby treating the disease or disorder that can benefit from increasing an M2/M1 macrophage ratio in the subject.

According to another aspect there is provided a therapeutically effective amount of basophils having been generated by culturing in the presence of IL33 and/or GM-SCF for use in treating a disease or disorder that can benefit from increasing an M2/M1 macrophage ratio in a subject in need thereof.

According to another aspect there is provided a method of treating a disease or disorder that can benefit from increasing an M2/M1 macrophage ratio in a subject in need thereof, the method comprising administering to the subject a therapeutically effective amount of a signaling molecule selected from the group consisting of IL6, IL13 and HGF, thereby treating the disease or disorder that can benefit from increasing an M2/M1 macrophage ratio in the subject.

According to another aspect there is provided a therapeutically effective amount of a signaling molecule selected from the group consisting of IL6, IL13 and HGF for use in treating a disease or disorder that can benefit from increasing an M2/M1 macrophage ratio in a subject.

As used herein “subject” refers to a subject suffering from a disease or disorder that can benefit from increasing an M1/M2 macrophage ratio or from a disease or disorder that can benefit from increasing an M2/M1 macrophage ratio. Alternatively, the subject is at a risk of developing such a disease or disorder.

When administering basophils, the cells can be autologus, non-autologous, allogeneic, syngeneic or xenogeneic (with the proper immune-suppression when needed).

As used herein “disease or disorder that can benefit from increasing M2/M1 macrophage ratio” refers to diseases or disorders (medical conditions in total) that can be ameliorated by suppressing the immune system.

Such typically include, but are not limited to, inflammation, autoimmunity, or injuries.

As used herein, the term “inflammatory disease” as used herein refers to acute or chronic localized or systemic responses to harmful stimuli, such as pathogens, damaged cells, physical injury or irritants, that are mediated in part by the activity of cytokines, chemokines, or inflammatory cells (e.g. macrophages) and is characterized in most instances by pain, redness, swelling, and impairment of tissue function. The inflammatory disease may be selected from the group consisting of: sepsis, septicemia, pneumonia, septic shock, systemic inflammatory response syndrome (SIRS), Acute Respiratory Distress Syndrome (ARDS), acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, cryoporin associated periodic syndromes and cirrhosis.

As used herein, an “autoimmune disease” is a disease or disorder arising from and directed at an individual's own tissues. Examples of autoimmune diseases include, but are not limited to Addison's Disease, Allergy, Alopecia Areata, Alzheimer's disease, Antineutrophil cytoplasmic antibodies (ANCA)-associated vasculitis, Ankylosing Spondylitis, Antiphospholipid Syndrome (Hughes Syndrome), arthritis, Asthma, Atherosclerosis, Atherosclerotic plaque, autoimmune disease (e.g., lupus, RA, MS, Graves' disease, etc.), Autoimmune Hemolytic Anemia, Autoimmune Hepatitis, Autoimmune inner ear disease, Autoimmune Lymphoproliferative syndrome, Autoimmune Myocarditis, Autoimmune Oophoritis, Autoimmune Orchitis, Azoospermia, Behcet's Disease, Berger's Disease, Bullous Pemphigoid, Cardiomyopathy, Cardiovascular disease, Celiac Sprue/Coeliac disease, Chronic Fatigue Immune Dysfunction Syndrome (CFIDS), Chronic idiopathic polyneuritis, Chronic Inflammatory Demyelinating, Polyradicalneuropathy (CIPD), Chronic relapsing polyneuropathy (Guillain-Barre syndrome), Churg-Strauss Syndrome (CSS), Cicatricial Pemphigoid, Cold Agglutinin Disease (CAD), chronic obstructive pulmonary disease (COPD), CREST syndrome, Crohn's disease, Dermatitis, Herpetiformus, Dermatomyositis, diabetes, Discoid Lupus, Eczema, Epidermolysis bullosa acquisita, Essential Mixed Cryoglobulinemia, Evan's Syndrome, Exopthalmos, Fibromyalgia, Goodpasture's Syndrome, Hashimoto's Thyroiditis, Idiopathic Pulmonary Fibrosis, Idiopathic Thrombocytopenia Purpura (ITP), IgA Nephropathy, immunoproliferative disease or disorder (e.g., psoriasis), Inflammatory bowel disease (IBD), including Crohn's disease and ulcerative colitis, Insulin Dependent Diabetes Mellitus (IDDM), Interstitial lung disease, juvenile diabetes, Juvenile Arthritis, juvenile idiopathic arthritis (JIA), Kawasaki's Disease, Lambert-Eaton Myasthenic Syndrome, Lichen Planus, lupus, Lupus Nephritis, Lymphoscytic Lypophisitis, Meniere's Disease, Miller Fish Syndrome/acute disseminated encephalomyeloradiculopathy, Mixed Connective Tissue Disease, Multiple Sclerosis (MS), muscular rheumatism, Myalgic encephalomyelitis (ME), Myasthenia Gravis, Ocular Inflammation, Pemphigus Foliaceus, Pemphigus Vulgaris, Pernicious Anaemia, Polyarteritis Nodosa, Polychondritis, Polyglandular Syndromes (Whitaker's syndrome), Polymyalgia Rheumatica, Polymyositis, Primary Agammaglobulinemia, Primary Biliary Cirrhosis/Autoimmune cholangiopathy, Psoriasis, Psoriatic arthritis, Raynaud's Phenomenon, Reiter's Syndrome/Reactive arthritis, Restenosis, Rheumatic Fever, rheumatic disease, Rheumatoid Arthritis, Sarcoidosis, Schmidt's syndrome, Scleroderma, Sjorgen's Syndrome, Stiff-Man Syndrome, Systemic Lupus Erythematosus (SLE), systemic scleroderma, Takayasu Arteritis, Temporal Arteritis/Giant Cell Arteritis, Thyroiditis, Type 1 diabetes, Type 2 diabetes, Ulcerative colitis, Uveitis, Vasculitis, Vitiligo, and Wegener's Granulomatosis.

As used herein “disease or disorder that can benefit from increasing an M1/M2 macrophage ratio” refers to diseases or disorders (medical conditions in total) that can be ameliorated by activating the immune system such as evidenced by the secretion of pro-inflammatory cytokines.

Such typically include, but are not limited to, cancer, e.g., metastatic cancer, progressive fibrotic diseases such as for example idiopathic pulmonary fibrosis (IPF), hepatic fibrosis systemic sclerosis, allergy and asthma, atherosclerosis and Alzheimer's disease, pulmonary fibrosis, liver fibrosis. In particularly, the method of the present invention is particularly suitable for the treatment of cancer. As used herein, the term “cancer” has its general meaning in the art and includes, but is not limited to, solid tumors and blood-borne tumors. The term cancer includes diseases of the skin, tissues, organs, bone, cartilage, blood and vessels. The term “cancer” further encompasses both primary and metastatic cancers. Examples of cancers that may be treated by methods and compositions of the invention include, but are not limited to, cancer cells from the bladder, blood, bone, bone marrow, brain, breast, colon, esophagus, gastrointestinal tract, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; Leydig cell tumor, malignant; lipid cell tumor, malignant; paraganglioma, malignant; extra-mammary paraganglioma, malignant; pheochromocytoma; glomangio sarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In some embodiments, the method of the present invention is particularly suitable for the treatment of metastatic cancer to bone, wherein the metastatic cancer is breast, lung, renal, multiple myeloma, thyroid, prostate, adenocarcinoma, blood cell malignancies, including leukemia and lymphoma; head and neck cancers; gastrointestinal cancers, including esophageal cancer, stomach cancer, colon cancer, intestinal cancer, colorectal cancer, rectal cancer, pancreatic cancer, liver cancer, cancer of the bile duct or gall bladder; malignancies of the female genital tract, including ovarian carcinoma, uterine endometrial cancers, vaginal cancer, and cervical cancer; bladder cancer; brain cancer, including neuroblastoma; sarcoma, osteosarcoma; and skin cancer, including malignant melanoma or squamous cell cancer.

The cells or agents (e.g., cytokines, growth factors, antibodies) of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the cells or agents (e.g., cytokines, growth factors, antibodies) accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient. According to a specific embodiment, the localized treatment is to the lung such as by intranasal administration.

Pulmonary administration cells or agents as described herein.

Pulmonary administration may be accomplished by suitable means known to those in the art. Typically, pulmonary administration requires dispensing of the biologically active substance from a delivery device into the oral cavity of a subject during inhalation. For example, compositions comprising cells or agents are administered via inhalation of an aerosol or other suitable preparation that is obtained from an aqueous or nonaqueous solution or suspension form, or a solid or dry powder form of the pharmaceutical composition, depending upon the delivery device used. Such delivery devices are well known in the art and include, but are not limited to, nebulizers, metered dose inhalers, and dry powder inhalers, or any other appropriate delivery mechanisms that allow for dispensing of a pharmaceutical composition as an aqueous or nonaqueous solution or suspension or as a solid or dry powder form. Methods for delivering cells or agents, to a subject via pulmonary administration, including directed delivery to the central and/or peripheral lung region(s), include, but are not limited to, a dry powder inhaler (DPI), a metered dose inhaler (MDI) device, and a nebulizer.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuos infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (cells or agents (e.g., cytokines, growth factors, antibodies)) effective to prevent, alleviate or ameliorate symptoms of a disorder (e.g., as described above) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide effective (e.g., the lung tissue) levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

As used herein the phrase “treatment regimen” refers to a treatment plan that specifies the type of treatment, dosage, schedule and/or duration of a treatment provided to a subject in need thereof (e.g., a subject diagnosed with a pathology). The selected treatment regimen can be an aggressive one which is expected to result in the best clinical outcome (e.g., complete cure of the pathology) or a more moderate one which may relief symptoms of the pathology yet results in incomplete cure of the pathology. It will be appreciated that in certain cases the more aggressive treatment regimen may be associated with some discomfort to the subject or adverse side effects (e.g., a damage to healthy cells or tissue). The type of treatment can include a surgical intervention (e.g., removal of lesion, diseased cells, tissue, or organ), a cell replacement therapy, an administration of a therapeutic drug (e.g., receptor agonists, antagonists, hormones, chemotherapy agents) in a local or a systemic mode, an exposure to radiation therapy using an external source (e.g., external beam) and/or an internal source (e.g., brachytherapy) and/or any combination thereof. The dosage, schedule and duration of treatment can vary, depending on the severity of pathology and the selected type of treatment, and those of skills in the art are capable of adjusting the type of treatment with the dosage, schedule and duration of treatment.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells - A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization - A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Mice

Sex- and age-matched Mcpt8-Cre^{+/-}DTA^{fl/+} and Mcpt8-Cre^{+/-}DTA^{+/+} littermate controls were used. YFP-expressing Mcpt8-Cre (B6.129-Mcpt8tml(Cre)Lksy/J) (Sullivan et al., 2011) and DTA (B6.129P2-Gt(ROSA) 26Sortml (DTA)Lky/J) (Voehringer et al., 2008) mice were kindly provided by Stephen Galli, Stanford University, and originally obtained from the Jackson Laboratory. Il1rl1^{-/-} (Townsend et al., 2000) mice were kindly provided by Andrew McKenzie, MRC Laboratory of Molecular Biology Cambridge. All these mice were bred and maintained at the animal facility of the Medical University of Vienna under specific pathogen free conditions. All experiments were performed in accordance with Austrian law and approved by the Austrian Federal Ministry of Sciences and Research (BMWFW-66.009/0146-WF/V/3b/2015). C57BL/6 WT pregnant, neonate and adult mice were obtained from Harlan. Mice were housed under specific-pathogen-free conditions at the Animal Breeding Center of the Weizmann Institute of Science. All animals were handled according to the regulations formulated by the Institutional Animal Care and Use Committee.

Tumor Cell Line

B 16F10 murine melanoma cells were maintained in DMEM, supplemented with 10% FCS, 100 U/mL penicillin, 100 mg/mL streptomycin and 1 mM 1-glutamine (Biological Industries). Cells were cultured in a humidified 5% CO2 atmosphere, at 37° C.

Method Details

Lung dissociation and single cell sorting

Single-cell experiments were performed on embryonic mouse lung at E12.5, E16.5, E18.5 and E19.5, on neonate lung at 1, 6, 7, 10, 16, 30h, 2 days, and 7 days PN, and on adult mouse lung (8-12 weeks). In general, embryonic experiments were performed on pooled sibling lungs of one litter (at E12.5 six lungs were pooled, at E16.5, E18.5 and E19.5 three lungs were pooled, at PN time points 2 lungs were pooled, and for adult lungs, samples were not pooled). Embryos were euthanized by laying on a frozen surface, while PN and adult mice were scarified by overdose of anesthesia. For all time points, except E12.5, mice were perfused by injection of cold PBS via the right ventricle prior to lung dissection. Lung tissue was dissected from mice and half tissues were homogenized using lung dissociation kit (Miltenyi Biotec), while enzymatic incubation was adapted to single cell protocol, and therefore was lasted 15 min (for 8 week adult mice, enzymatic digestion was lasted 20 min). The second half of the lung was dissociated as previously documented (Treutlein et al., 2014), briefly cells were supplemented with DMEM/F12 medium (Sigma-Aldrich) containing Elastase (3 U/ml, Worthington) and

DNase (0.33 U/ml, Sigma-Adrich) incubated with frequent agitation at 37° C. for 15 min. Next, an equal volume of DMEM/F12 supplemented with 10%FBS, 1 U/ml penicillin, and 1Uml streptomycin (Biological Industries) was added to single-cell suspensions. Following dissociations, single cell suspension of the same lung was merged and centrifuged at 400 g, 5 min, 4° C. All samples were filtered through a 70 μm nylon mesh filter into ice cold sorting buffer (PBS supplemented with 0.2mM EDTA pH8 and 0.5% BSA).

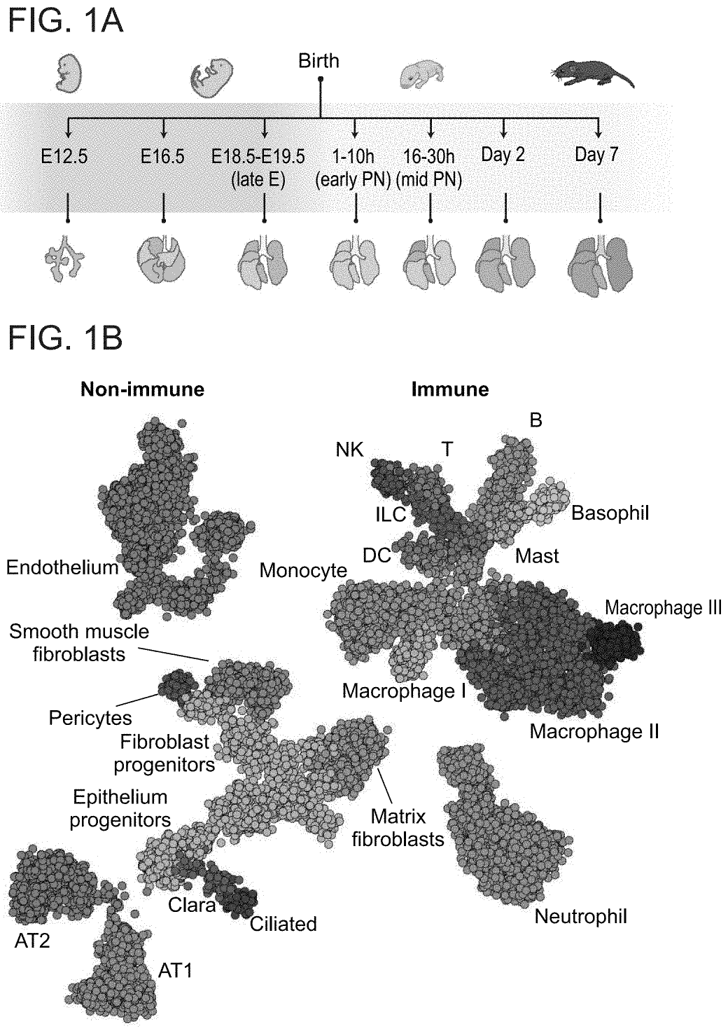

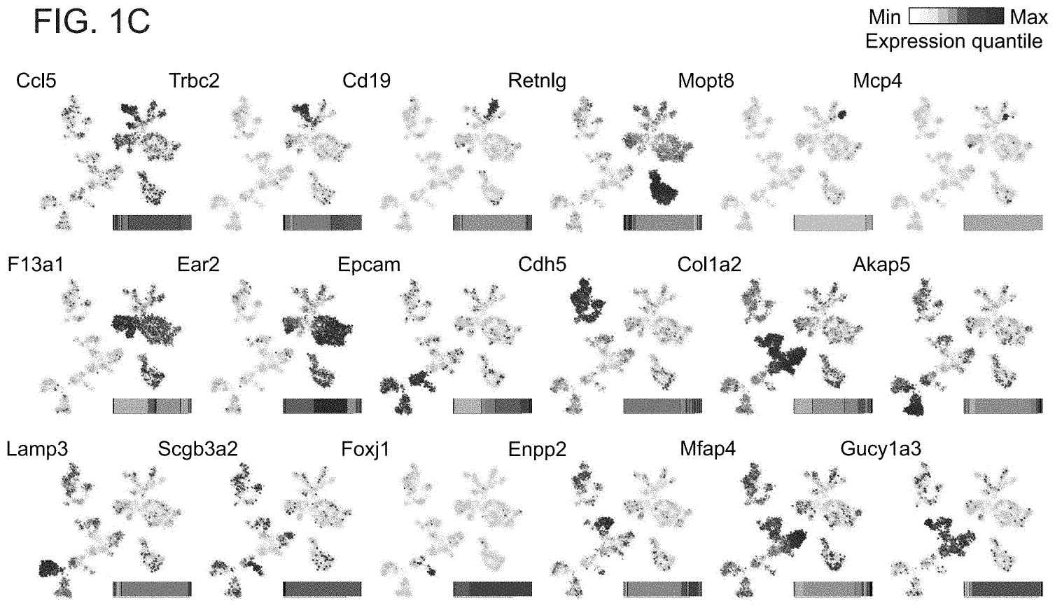

For calibration of lung dissociation protocol, cells derived from adult mouse lungs were supplemented with 1). DMEM (Biological Industries) containing Liberase (50 μg/ml, Sigma-Aldrich) and DNase (1 μg/ml, Roche); 2). PBS Ca⁺Mg⁺(Biological Industries) containing Collagenase IV (1 mg/ml, Worthington) and Dispase (2.4 U/ml, Sigma-Adrich); 3). DMEM/F12 (Sigma-Aldrich) containing Elastase and DNase, as described above; and 4). Enzymes derived from lung dissociation kit (Miltenyi biotec), as described above. Following enzymatic digestion with frequent agitation at 37° C. for 20 min, an equal volume of DMEM supplemented with 10% FBS, 1 U/ml penicillin, and 1Uml streptomycin (Biological Industries), or sorting buffer was added to single-cell suspensions from liberase and collagenase-dispase treatments, respectively. All live cells were sorted, after exclusion of doublets and erythrocytes, for MARS-seq analysis. Single cell analysis of cells extracted by each dissociation technique showed differential distribution of cell types (not shown). Next, we chose dissociation protocol for the study that extracted vast range of cell populations from the immune and the non-immune compartments, without any preference to specific cell type stemming from the dissociation enzymes. Therefore, lung digestions along the study were a combination of elastase digestion, which lead to the extraction of epithelial cells and AM, and miltenyi kit protocol, which led to the extraction of different cell populations from the immune compartment. Importantly, these digestions were not characterized in any cell type preference, like endothelium dominancy that we found following collagenase-dispase and liberase treatments (not shown); however, the percentages of cells observed in the single cell maps are dependent on the different lung dissociation methods (FIG. 1B, 2B-C).

Isolation of Peripheral Blood Cells

Peripheral blood cells were suspended with 200 of heparin, and washed with PBS supplemented with 0.2mM EDTA pH8 and 0.5% BSA. Cells were suspended with ficoll-Paque™ PLUS (1:1 ratio with PBS, Sigma-Adrich) and centrifuged at 460 g, 20 min, 10° C., with no-break and no-acceleration. The ring-like layer of mononuclear cells was transferred into new tube and washed twice with cold PBS, centrifuged at 400 g, 5 min, 4° C., passed through a 40ᵢ.tm mesh filter, and then suspended in ice-cold sorting buffer.

Tumor Microenvironment Dissociation

For purification of basophils from tumor microenvironment, 1×10⁶ cells were suspended in 1000 μl PBS and injected subcutaneous (s.c.) into 8-week mice. Solid tumors were harvested 10 days post injection, cut into small pieces, and suspended with RPMI-1640 supplemented with DNase (12.5 μg/ml, Sigma-Adrich) and collagenase IV (1 mg/ml, Worthington). Tissues were homogenized by GentleMacs tissue homogenizer (Miltenyi Biotec), and incubated at 37° C. for 10 min. Following two times of mechanic and enzymatic dissociation, cells were washed and suspended in red blood lysis buffer (Sigma-Aldrich) and DNase (0.33 U/ml, Sigma-Adrich), incubated for 5 min at room temperature, washed twice with cold PBS, passed through a 40 μm mesh filter, centrifuged at 400 g, 5 min, 4° C. and then resuspended in ice cold sorting buffer.

Spleen Dissociation

Tissue was harvested from 8 week females, suspended with accutase solution (Sigma-Adrich), homogenized by GentleMacs tissue homogenizer (Miltenyi Biotec), and incubated with frequent agitation at 37° C. for 10 min. Cells were washed and suspended in red blood lysis buffer (Sigma-Aldrich) and DNase (0.33 U/ml, Sigma-Adrich), incubated for 3 min at room temperature, washed twice with cold PBS, passed through a 40 μm mesh filter, centrifuged at 400 g, 5 min, 4° C. and then resuspended in ice cold sorting buffer.

Liver Dissociation

Basophils from the liver were isolated by a modification of the two-step collagenase perfusion method of Seglen (Seglen, 1973). Digestion step was performed with Liberase (20μg/ml; Roche Diagnostics) according to the manufacturer's instruction. Liver was minced to small pieces, suspended with PBS and centrifuged at 30 g, 5 min, 4° C. Supernatant was collected in new tube (to remove hepatocytes), suspended with PBS and centrifuged at 30 g, 5 min, 4° C. (this step was repeated twice). Following second wash, supernatant was collected in new tube, centrifuged at 500 g, 5 min, 4° C., and then resuspended in ice-cold sorting buffer.

Flow Cytometry and Sorting

Cell populations were sorted with SORP-aria (BD Biosciences, San Jose, Calif.) or with AriaFusion instrument (BD Biosciences, San Jose, Calif.). Samples were stained using the following antibodies: eF780-conjugated Fixable viability dye, eFluor450-conjugated TER-119, APC-conjugated CD45, FITC-conjugated CD117 (cKit), and PerCPCy5.5-conjugated F4/80 were purchased from eBioscience, PerCP Cy5.5-conjugated FCERal (MARI), APC-Cy7-conjugated Ly6G, FITC-conjugated CD3, PE-Cy7-conjugated CD19, PE-Cy7-conjugated CD31, APC-Cy7-conjugated CD326, APC/Cy7-conjugated TER-119, AF700-conjugated CD45, Pacific blue-conjugated CD49b, PE-conjugated Fcer1 a, PE/Cy7-conjugated CD117, FITC-conjugated Ly6C, PE-conjugated CD11c, BV605-conjugated CD11b and BV605-conjugated Ly-6C were purchased from Biolegend, and FITC-conjugated CD11C was purchased from BD-Pharmingen. Prior to sorting, cells were stained with DAPI or fixable viability dye for evaluation of live/dead cells, and then filtered through a 40 μm mesh. For the sorting of whole immune cell populations, samples were gated for CD45⁺, for sorting of whole stromal cell samples were gated for CD45⁻, and for the isolation of basophils, samples were gated for CD45⁺FCεR1α⁶³⁰ cKit⁻, after exclusion of doublets, dead cells and erythrocytes. To record marker level of each single cell, the FACS Diva 7 “index sorting” function was activated during single cell sorting. Following the sequencing and analysis of the single cells, each surface marker was linked to the genome-wide expression profile. This methodology was used to optimize the gating strategy. Isolated live cells were single-cell sorted into 384-well cell capture plates containing 2ₗit of lysis solution and barcoded poly(T) reverse-transcription (RT) primers for single-cell RNA-seq (Jaitin et al., 2014; Paul et al., 2015). Four empty wells were kept in each 384-well plate as a no-cell control during data analysis. Immediately after sorting, each plate was spun down to ensure cell immersion into the lysis solution, and stored at −80° C. until processed.

For evaluation of protein levels of receptors expressed by lung basophils, we performed cell surface staining of PE-conjugated CD131 (CSF2Rb, Miltenyi Biotec), PE/Cy7-conjugated IL-33R (Biolegend), and PacificBlue-conjugated CD49b (Biolegend). For evaluation of intracellular protein levels of ligands expressed by lung basophils, cells were incubated with RPMI-1640 supplemented with 10% FCS, 1mM 1-glutamine, 100 U/ml penicillin, 100 mg/ml streptomycin (Biological Industries) and GolgiStop (1:1000; for IL-13, BD bioscience, San Jose, Calif.), or Brefeldin A solution (1:1000, for IL-6, Biolegend), for 2h at 37° C., to enable expression of intracellular cytokines, and to prevent their extracellular secretion. Cells were washed, fixed, permeabilized and stained for surface and intracellular proteins using the Cytofix/Cytoperm kit, according to the manufacture's instructions (BD bioscience, San Jose, Calif.). For the intracellular experiments the following antibodies were used: PE-conjugated IL-6 (Biolegend), PE-conjugated IL-13 (eBioscience) and matched Isotype control PE-conjugated Rat IgG1 (Biolegend). Cells were analyzed using BD FACSDIVA software (BD Bioscience) and FlowJo software (FlowJo, LLC).

BM derived cell cultures

BM progenitors were harvested from C57BL/6 8 week old mice and cultured at concentration of 0.5×10⁶ cells/ml. For BM-MΦ differentiation, BM cultures were cultured for 8 days in the presence of M-CSF (50 ng/ml; Peprotech). On day 8, cells were scraped with cold

PBS and replated on 96-well flat bottom tissue culture plates for 16h. For BM-derived basophils differentiation, BM cultures were cultured for 10 days in the presence of IL-3 (30 ng/ml; Peprotech). Basophils were enriched by magnetic-activated cell sorting for CD117⁻population (cKit; Miltenyi Biotec), and replated on 96-well flat bottom tissue culture plates for 16h. All BM cultures were done in the standard media RPMI-1640 supplemented with 10% FCS, 1mM 1-glutamine, 100 U/ml penicillin, 100 mg/ml streptomycin (Biological Industries). Every 4 days BM cultures were treated with differentiation factors M-CSF (50 ng/ml) or IL-3 (30 ng/ml). Following replating of BM-derived cells, co-cultured and mono-cultured cells were seeded in concentration of 0.5×10⁶ cells/ml (1:1 ration in co-cultures), and supplemented with IL-3 (10 ng/ml) and M-CSF (lOng/ml) for cell survival, IL33 (50 ng/ml; Peprotech) or GM-CSF (50 ng/ml; Peprotech) for cell activation.

For co-culture of BM-basophils with lung-derived monocytes and undifferentiated macrophages, we sorted CD45⁺CD115⁺ myeloid cells from 30h PN lungs and performed the in vitro experiment, as detailed above.

MARS-Seq Library Preparation

Single-cell libraries were prepared as previously described (Jaitin et al., 2014). In brief, mRNA from cell sorted into cell capture plates were barcoded and converted into cDNA and pooled using an automated pipeline. The pooled sample is then linearly amplified by T7 in vitro transcription, and the resulting RNA is fragmented and converted into a sequencing-ready library by tagging the samples with pool barcodes and illumina sequences during ligation, RT, and PCR. Each pool of cells was tested for library quality and concentration is assessed as described earlier (Jaitin et al., 2014).

Lung-Resident Basophil Depletion

For depletion of basophils in neonate lungs, we calibrated a protocol based on previous studies (Denzel et al., 2008; Guilliams et al., 2013). Mice were injected i.n. with 7 μl of 100 μg anti-Fcεr1α (MARI; eBioscience) or IgG isotype control (Armenian hamster, eBioscience) twice, at 10h and 15h following birth. Lungs were purified from injected neonates 30h following birth and CD45⁺ cells were sorted for RNA-seq analysis.

Phagocytosis Assay

Phagocytosis assays were performed as described earlier (Sharif et al., 2014). AM were isolated by bronchoalveolar lavage (BAL). In brief, the trachea of mice was exposed and cannulated with a sterile 18-gauge venflon (BD Biosciences) and 10m1 of sterile saline were instilled in 0.5m1 steps. Total cell numbers in the retrieved BAL fluid (comprising >95% AM) were counted using a Neubauer chamber. To assess bacterial phagocytosis, 1-2.5×10⁵ AM were plated and allowed to adhere for 3h in RPMI containing 10% fetal calf serum (FCS), 1% penicillin and 1% streptomycin. Next, AM were incubated with FITC-labeled heat-inactivated S. pneumoniae (MOI 100) for 45 min at 37° C. or 4° C. (as a negative control). Cells were washed and incubated with proteinase K (50 m/ml) for 10 min on ice to remove adherent bacteria. Uptake of bacteria was assessed via flow cytometry and the phagocytosis index was calculated as (MFI× % positive cells at 37° C.) minus (MFI× % positive cells at 4° C.).

Single-Molecule Fluorescent in situ Hybridization (smFISH)

Neonates in the age of 7 days were perfused with PBS. Lung tissues harvested and fixed in 4% paraformaldehyde for 3h at 4° C., incubated overnight with 30% sucrose in 2% paraformaldehyde at 4° C. and then embedded in OCT. Cryo-sections (6 μm) were used for hybridization. Probe libraries were designed and constructed as previously described (Itzkovitz et al., 2012, Stellaris Fish Probes # SMF-1082-5, SMF-1063-5, SMF 1065-5). Single molecule FISH probe libraries consisted of 48 probes of length 20 bps. smFISH probe libraries of Il1r11, Il33, and Mcpt8 probes were coupled to Cy3, AF594, and cy5, respectively. Hybridizations were performed overnight in 30° C. DAPI dye for nuclear staining was added during the washes. Images were taken with a Nikon Ti-E inverted fluorescence microscope equipped with a x60 and x100 oil-immersion objective and a Photometrics Pixis 1024 CCD camera using MetaMorph software (Molecular Devices, Downington, Pa.). smFISH molecules were counted only within the DAPI staining of the cell.

Histology and Immunohistochemistry

For histologic examination, paraffin-embedded lung sections were taken at indicated time-points. To stain for proSP-C, endogenous peroxidase activity was quenched and antigen was retrieved with Antigen Unmasking Solution (Vector Laboratories, H-3300). Blocking was done in donkey serum and the slides were then stained with anti-proSP-C (Abcam), followed by secondary goat-anti-rabbit IgG antibody (Vector Laboratories), and signal amplification using the Vectastain ELITE kit (Vector Laboratories). For F4/80 staining, antigen was retrieved using protease type XIV (SIGMA), followed by blocking with rabbit serum and staining with rat-anti-mouse F4/80 mAb (AbD Serotec). A secondary rabbit-anti-rat IgG Ab (Vector Laboratories) was applied and the signal was amplified with Vectastain ELITE kit (Vector Laboratories). For Mcpt8 staining, an anti-GFP Ab (Abcam) was used followed by a secondary biotinylated rabbit-anti-goat IgG Ab (Vector Laboratories). For detection, Peroxidase Substrate kit (Vector) or Vector VIP Peroxidase Kit (Vector Laboratories) was applied. Cell structures were counter-stained with hematoxylin or methylgreen and pictures were taken on an Olympus FSX100 Microscope.

For whole lobe analysis, slides were scanned using a TissueFAXS imaging system (TissueGnostics GmbH) equipped with a Zeiss Axio Imager.Z1 microscope (Carl Zeiss Inc., Jena, Germany). Images were taken using a PCO PixelFly camera (Zeiss).

Tissue Clearing

Tissue clearing protocol was performed as described earlier (Fuzik et al., 2016). In short, lungs at indicated time-points were perfused once with PBS and afterwards with 7.5% formaldehyde in PBS. Lung lobes were fixed in 7.5% formaldehyde in PBS at room temperature overnight. Lung lobes were cleared using CUBIC reagent 1 (25 wt % urea, 25 wt % N,N,N′,N′-tetrakis(2-hydroxypropyl) ethylenediamine and 15 wt % Triton X-100) for 4 days (30h PN, day 8.5) or 7 days (8-weeks) at 37° C. After repeated washes in PBS, lung lobes were incubated in blocking solution (PBS, 2.5% BSA, 0.5% Triton X-100, 3% normal donkey serum) and afterwards placed in primary antibody solution (1:100; goat anti-mouse GFP, abcam) for 4 days (30h PN, day 8.5) or 5 days (8-weeks) at 37° C. After washing the secondary antibody solution (1:500; donkey anti-goat AF555, Invitrogen) was added for 4 days (30h PN, day 8.5) or 5 days (8-weeks) at 37° C. After re-washing with PBS and a fixing step for 2h at room temperature in 7.5% formaldehyde, washing steps were repeated and lung lobes were incubated in CUBIC reagent 2 (50 wt % sucrose, 25 wt % urea, 10 wt % 2,20,20′-nitrilotriethanol and 0.1% v/v % Triton X-100) for another 4 days (30h PN, day 8.5) or 7 days (8-weeks). Cleared lung lobes were imaged in CUBIC reagent 2 with a measured refractive index of 1.45 using a Zeiss Z1 light sheet microscope through 5× detection objective, 5× illumination optics at 561 laser excitation wavelength and 0.56× zoom. Z-stacks were acquired in multi-view tile scan mode by dual side illumination with light sheet thickness of 8.42 μm and 441.9 ms exposure. Stitching, 3D reconstruction, visualization and rendering was performed using Arivis Vision4D Zeiss Edition (v.2.12).

Quantification and Statistical Analysis

Low Level Processing and Filtering

All RNA-Seq libraries (pooled at equimolar concentration) were sequenced using Illumina NextSeq 500 at a median sequencing depth of 58,585 reads per single cell. Sequences were mapped to mouse genome (mm9), demultiplexed, and filtered as previously described (Jaitin et al., 2014), extracting a set of unique molecular identifiers (UMI) that define distinct transcripts in single cells for further processing. We estimated the level of spurious UMIs in the data using statistics on empty MARS-seq wells (median noise 2.7%; not shown). Mapping of reads was done using HISAT (version 0.1.6) (Kim et al., 2015); reads with multiple mapping positions were excluded. Reads were associated with genes if they were mapped to an exon, using the UCSC genome browser for reference. Exons of different genes that shared genomic position on the same strand were considered a single gene with a concatenated gene symbol. Cells with less than 500 UMIs were discarded from the analysis. After filtering, cells contained a median of 2,483 unique molecules per cell. All downstream analysis was performed in R.

Data Processing and Clustering

The Meta-cell pipeline (Giladi et al., 2018) was used to derive informative genes and compute cell-to-cell similarity, to compute K-nn graph covers and derive distribution of RNA in cohesive groups of cells (or meta-cells), and to derive strongly separated clusters using bootstrap analysis and computation of graph covers on resampled data. A full description of the method and downstream analysis is depicted in Figures. Default parameters were used unless otherwise stated.

Clustering of lung development was performed for the immune (CD45⁺) and non-immune (CD45⁻) compartments combined. Cells with high (>64) combined expression of hemoglobin genes were discarded (Hba-a2, Alas2, Hba-a1, Hbb-b2, Hba-x, Hbb-b1). We used bootstrapping to derive robust clustering (500 iterations; resampling 70% of the cells in each iteration, and clustering the co-cluster matrix with minimal cluster size set to 20). No further filtering or cluster splitting was performed on the meta-cells.

In order to annotate the resulting meta-cells into cell types, we used the metric FP - gene,mc (not shown), which signifies for each gene and meta-cell the fold change between the geometric mean of this gene within the meta-cell and the median geometric mean across all meta-cells. The FP metric highlights for each meta-cell genes which are robustly over-expressed in it compared to the background. We then used this metric to “color” meta-cells for the expression of lineage specific genes such as Clic5 (AT1), Ear2 (macrophages), and Cd79b (B cells), etc. Each gene was given a FP threshold and a priority index—such that coloring for AT1 by Clic5 is favored over coloring for general epithelium by Epcam. The selected genes, priority, and fold change threshold parameters are as follows:

TABLE 3

| | | | fold |
|---|---|---|---|
| group | gene | priority | change |
| Epithel | Epcam | 1 | 2 |
| AT1 | Clic5 | 3 | 5 |
| AT2 | Sftpc | 3 | 40 |
| Endothel | Cdh5 | 4 | 4 |
| Fibro | Co11a2 | 1 | 2 |
| Pericytes | Gucy1a3 | 3 | 5 |
| Club | Scgb3a2 | 3 | 2 |
| Matrix | Mfap4 | 3 | 10 |
| Smooth | Tgfbi | 2 | 8 |
| Ciliated | Ccdc19 | 3 | 2 |
| Ciliated | Foxj1 | 3 | 2 |
| B | Cd79b | 1 | 2 |
| Baso | Mcpt8 | 5 | 2 |
| DC | Flt3 | 4 | 2 |
| MacI | Cx3cr1 | 4 | 6 |
| MacII | Ear2 | 3 | 2 |
| MacIII | Ccl6 | 5 | 20 |
| MacIII | Cd9 | 5 | 7 |
| Mast | Mcpt4 | 4 | 2 |
| Mast | Gata2 | 3 | 3 |
| Mon | Ccr2 | 2 | 2 |
| Mon | F13a1 | 3 | 4 |
| Mon | Fcgr4 | 5 | 3.5 |
| Mon | Csf1r | 3 | 4 |
| Neut | S100a8 | 1 | 20 |
| Neut | Csf3r | 4 | 5 |
| NK | Gzma | 3 | 5 |
| T | Trbc2 | 2 | 2 |
| ILC | Rora | 4 | 2 |

Trajectory Finding

To infer trajectories and align cells along developmental pseudo-time, we used the published package Slingshot (Street et al., 2017). In short, Slingshot is a tool that uses pre-existing clusters to infer lineage hierarchies (based on minimal spanning tree, MST) and align cells in each cluster on a pseudo-time trajectory. Since our data is complex and contains many connected components and time points, we chose to apply Slingshot on subsets of interconnected cells type, namely E16.5 monocytes and macrophage II and III (dataset a), and the fibroblast lineage (dataset b).

For dataset a, we performed Slingshot on all macrophages II-III and on monocyte meta-cells with low relative expression of Ly6c2 (excluding differentiated monocytes and retaining E16.5 monocytes). For each dataset we chose a set of differential genes between the cell types (FDR corrected chi²test, q<10⁻³, fold change>2). We performed PCA on the log transformed UMI normalized to cell size. We ran Slingshot on the seven top principal components, with monocytes and early fibroblasts as starting clusters.

We first observe strong AT1 and AT2 signatures on day E18.5. This is parallel to disappearance of progenitor epithelium cells. From this we hypothesized that the precise branching point is not sampled with high temporal resolution in our developmental cohort, rendering Slingshot inefficient for this particular case. Instead, we examined whether progenitor epithelial cells on day E16.5 may be already primed toward either AT1 or AT2. To detect AT1 AT2 priming in epithelium progenitors, we used published gene lists of AT1 and AT2 (Treutlein et al., 2014) and computed two scores by the following term: Σ_{gene}log(1+7*UMI_{gene}^{cell}). We then examined score distribution in epithelium progenitors.

Interaction Maps

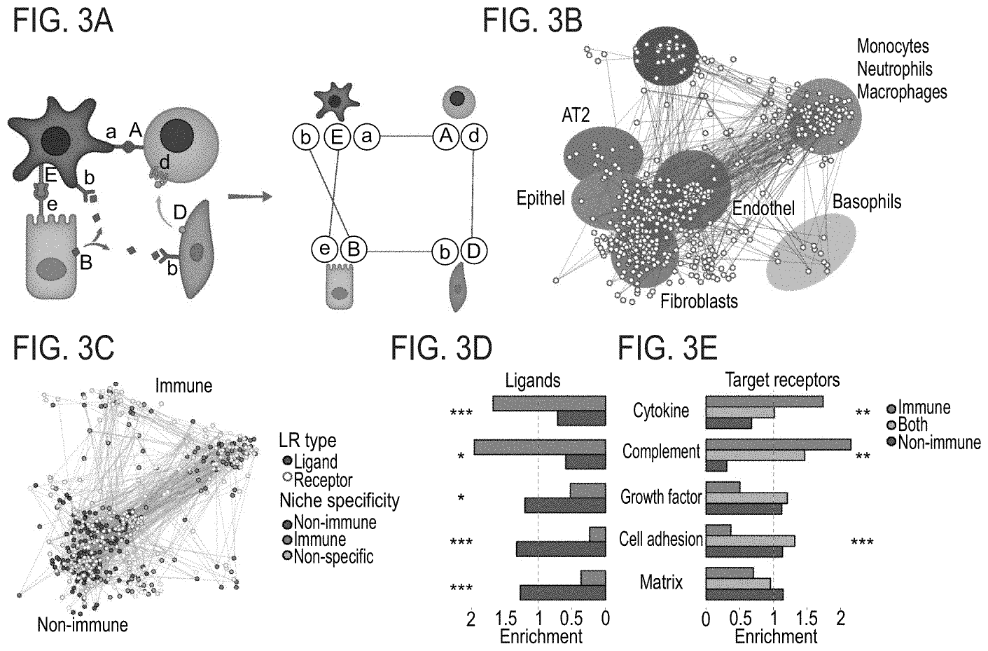

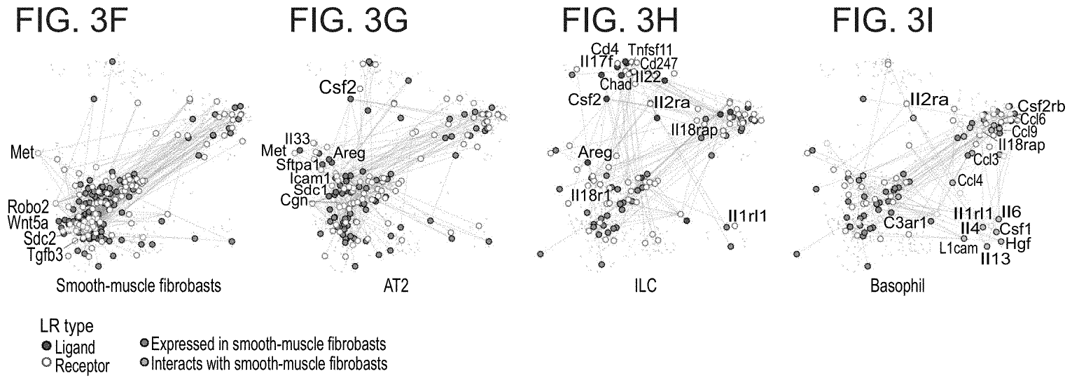

To visualize all lung interactions, we used a published dataset of ligand and receptor pairs (Ramilowski et al., 2015). We applied a lenient filtering, including all LR with >13 UMI in at least one meta-cell (normalized to meta-cell size). We computed the Spearman correlation between the log transformed UMI (down-sampled to 1000 UMI), and used hierarchical clustering to identify LR modules (cutree with K=15). We built a scaffold of an interaction graph by computing the Spearman correlation between LR modules and connecting edges between modules with ρ>0.4, generating a graph with the Rgraphviz package. We projected single LR on the graph scaffold by computing the mean x,y coordinates across all LR with ρ>0.05 (FIG. 3B).

To determine enrichment of stroma-stroma and immune-immune interactions we determined for each LR whether it's mainly expressed in the stromal or the immune compartments (log2 fold change >1, not shown). We computed the number of S-S and I-I interactions and compared to 10,000 randomly generated graphs. Importantly, as the interaction graph is not regular, we preserved nodes' degrees for each randomly generated graph. Ligand functional groups were extracted from David GO annotation tool (Huang da et al., 2009), and curated manually.

For projections in FIG. 3E-H, a cell type was determined to express a LR if its expression was more than two fold higher than in all other cells.

Mapping Cells to the Lung Cluster Model

Given an existing reference single cell dataset and cluster model, and a new set of single cell profiles, we extract for each new cell the K (K=10) reference cells with top Pearson correlation on transformed marker gene UMIs as described above. The distribution of cluster memberships over these K-neighbors was used to define the new cell reference cluster (by majority voting).

Basophil Profiling, Ex Vivo and Co-Culture Analysis

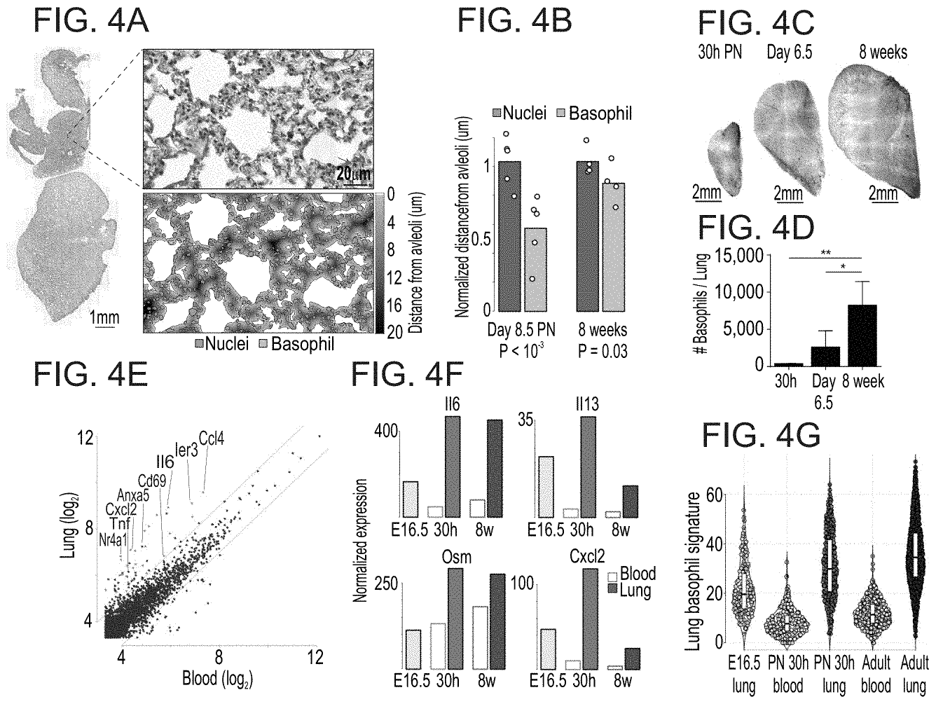

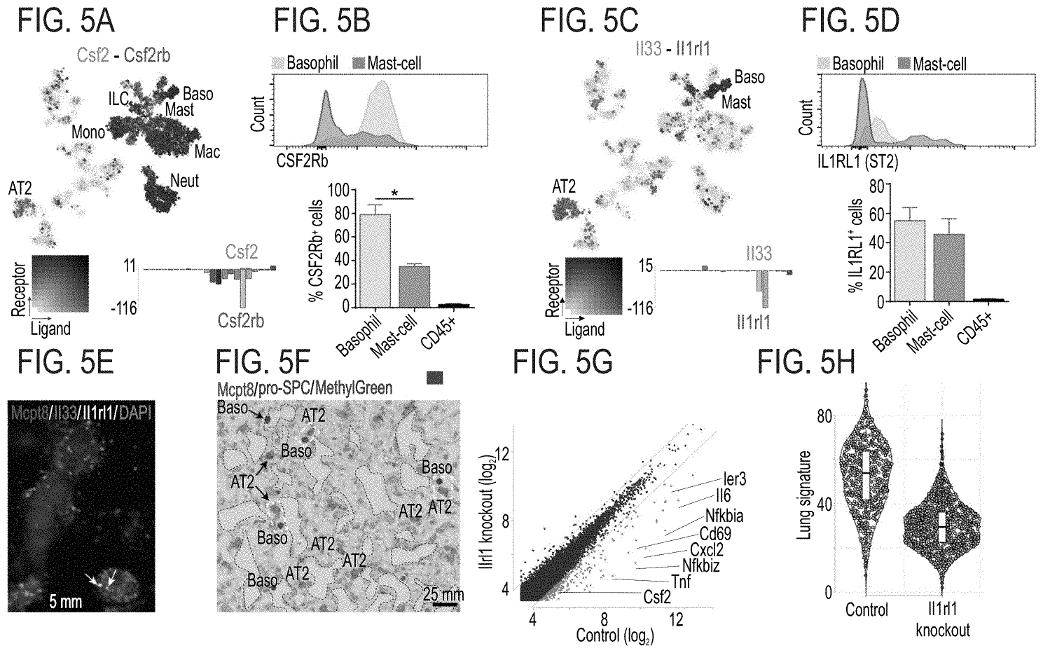

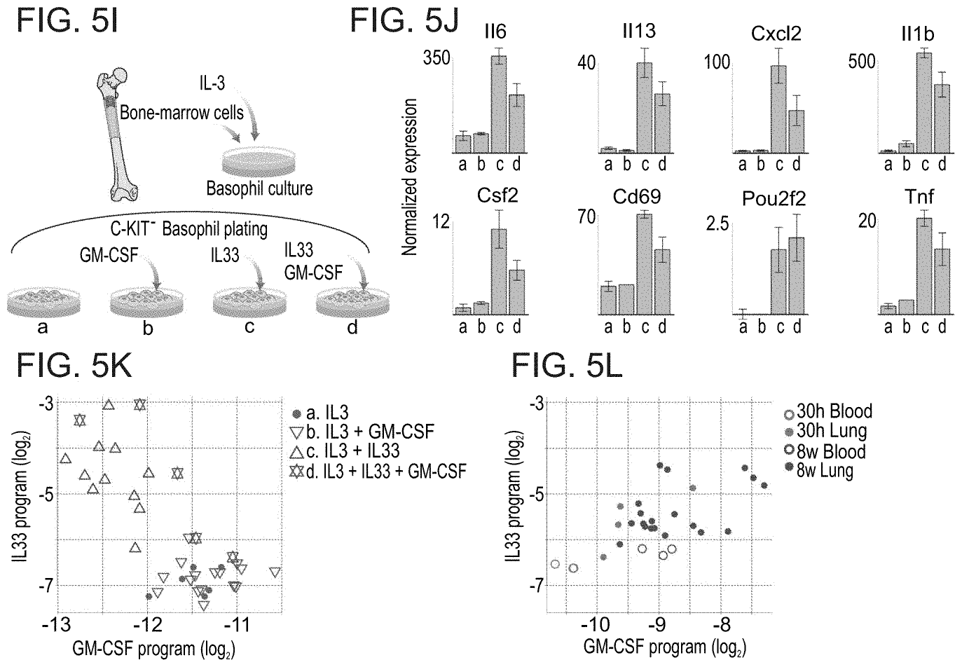

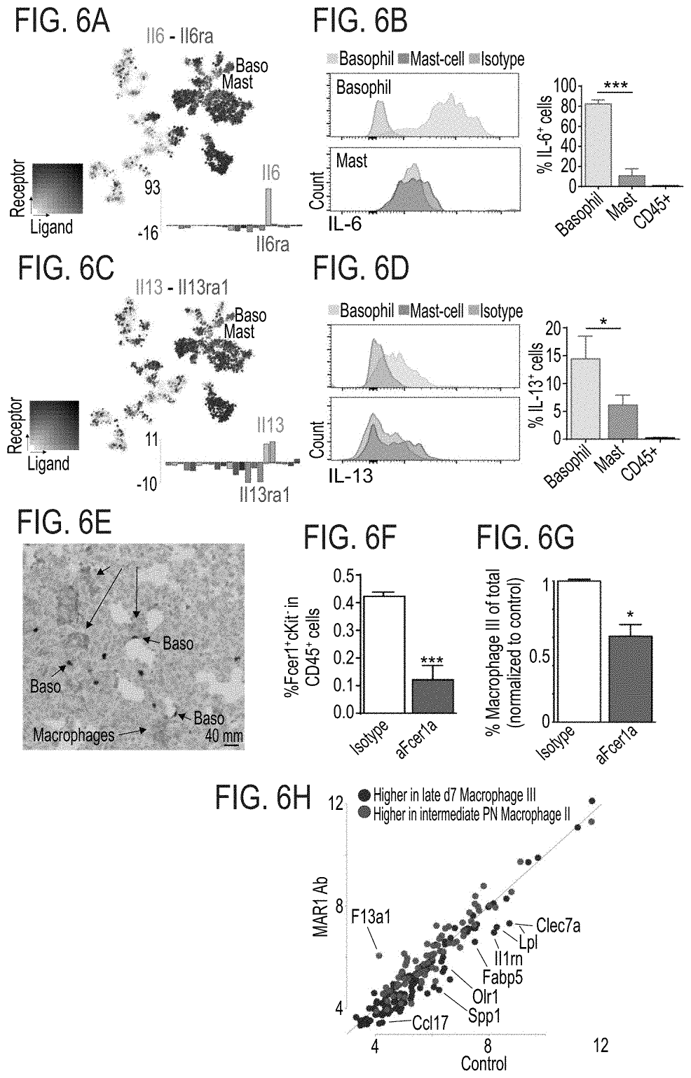

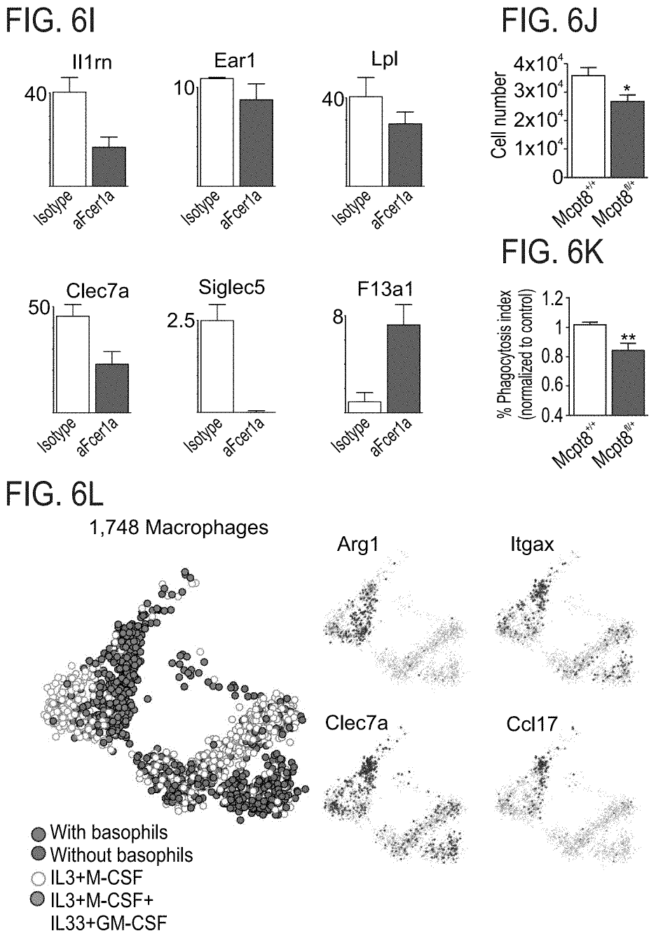

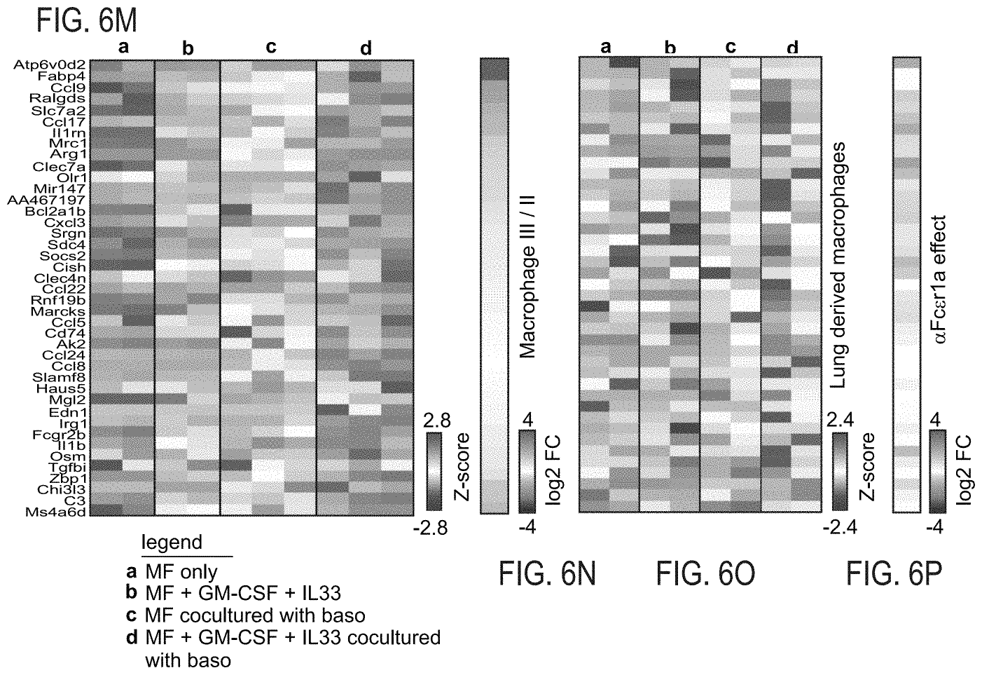

We used the MetaCell pipeline to analyze and filter the following datasets: (a) lung and blood derived basophils (FIGS. 4E-G); (b) Il1rl1 knockout and control (FIGS. 5G-H); (c) ex vivo grown basophils (FIGS. 5J-L, S5D); (d) and ex vivo co-culture of macrophages and basophils (FIGS. 6L-M, S6J). Meta cell analysis was performed with default settings. In each dataset we identified basophils and filtered contaminants by selecting meta-cells with increased mean expression of Mcpt8 against the median. In the co-culture experiment (d), meta-cells were determined as macrophages by increased mean expression of Csf1r.

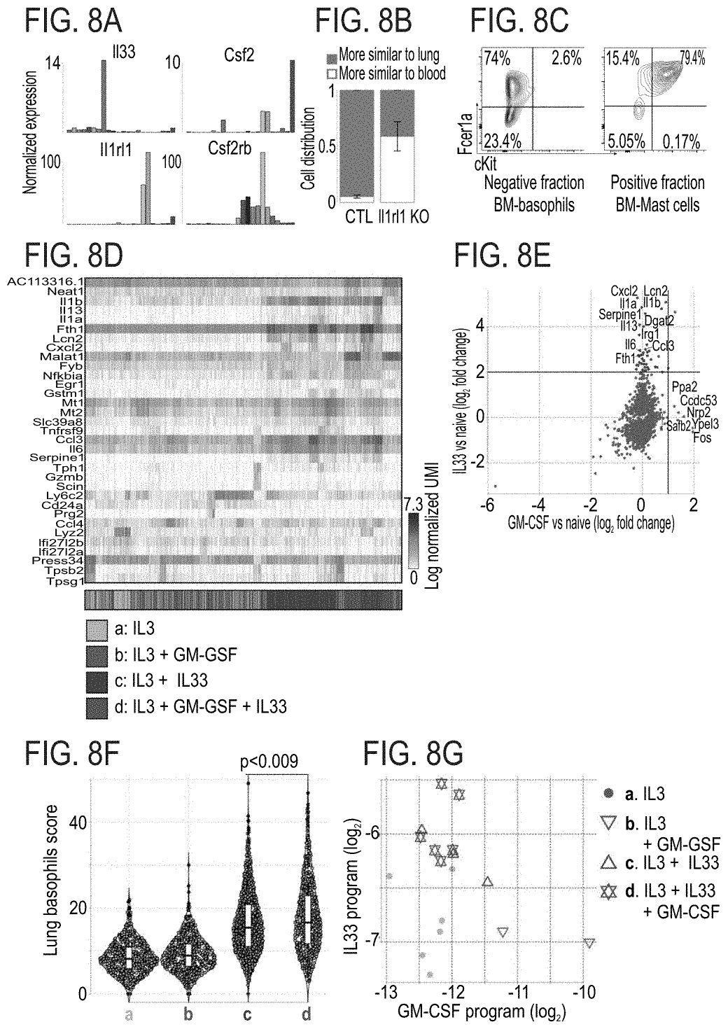

To compute the combined expression of genes in single cells (FIGS. 8A-G), we computed the following term: Σ_{gene}log(1+7*UMI_{gene}^{cell}). This allows pooling of gene at different expression levels.

TissueFAXS Quantification

TissueFAXS images were processed by MATLAB (R2014b). Segmentation of alveoli was performed by a custom-made pipeline. Images were converted to grayscale and enhanced, opened and closed with a disk size of 15 pixels. Alveoli were determined by intensity threshold of 200. Areas larger than 300,000 pixels were discarded. Segmentation of nuclei was performed by a similar pipeline (disk size=5 pixels), followed by applying a watershed algorithm, and detection of local minima. Images were converted to L*A*B color-space, and mean values of each nucleus were collected. Nuclei at the edges of the section were discarded. Nuclei with area <Tₐᵣₑₐ, mean luminance >T₁ or high circularity score (>T_{circ}) were discarded. Nuclei distances to alveoli (in pixels) were calculated with the bwdist method. Basophils (which are YFP⁺) are distinguished from other nuclei by their dark brownish hue (FIG. 4A). Therefore, we identified basophils by having low mean luminance and high mean b color channel (mean(b)-mean(1)>T_{baso}). For day 8.5 PN lobes we used the following parameters: Tₐᵣₑₐ=50; T₁=60; T_{circ}=5; T_{baso}=−40. For 8 weeks lobes we used the following parameters: Tₐᵣₑₐ=20; T₁=60; T_{circ}=5; T_{baso}=−40. To validate that our results are not affected by low quality sections, we randomly selected subsections from each TissueFAXS lobe, and manually inspected them for image clarity. We repeated until we obtained at least 200 basophils per lobe, or until no more basophils existed in lobe. We tested for significance of distances to alveoli as follows: For each lobe we rank-transformed all nuclei distances separately. We then randomly selected N_{baso} nuclei from each lobe (where N_{baso} stand for the number of basophils in that lobe), and calculated the median ranked distance. We repeated this permutation process 10⁵ times for each time point and compared them to the observed median ranked distances.

Data and Software Availability All reported data will be uploaded and stored in GEO, accession number GSE119228. Software and custom code will be available by request.

### Example 1

A comprehensive map of the lung cell types during development To understand the contribution of different immune and non-immune cell types and states for lung development and homeostasis, we collected single cell profiles along critical time points of lung development. In order to avoid biases stemming from cell-surface markers or selective tissue dissociation procedures, we combined a broad gating strategy and permissive tissue dissociation protocol, resulting in a comprehensive repertoire of the immune and non-immune cells located in the lung (not shown; Methods). We densely sampled cells from multiple time points of lung embryonic and postnatal development, and performed massively parallel single cell RNA-seq coupled to index sorting (MARS-seq) (Jaitin et al., 2014) (FIG. 1A; and not shown). We collected cells from major embryonic developmental stages: early morphogenesis (E12.5), the canalicular stage (E16.5) and the saccular stage (E18.5 - E19.5; Late E). We further collected cells from postnatal stages of alveolarization immediately after birth (1,6,7 and 10h postnatal; Early PN), 16 and 30h postnatal (Mid PN), as well as 2 days and 7 days postnatal (FIG. 1A). To construct the lung cellular map, we profiled 10,196 CD45⁻ (non-immune) and 10,904 CD45⁺ (immune) single cells from 17 mice and used the MetaCell algorithm to identify homogeneous and robust groups of cells (“meta-cells”; Methods) (Giladi et al., 2018), resulting in a detailed map of the 260 most transcriptionally distinct subpopulations (not shown). A two-dimensional representation of immune and non-immune single cells revealed separation of cells into diverse lineages (FIG. 1B). In the immune compartment, lymphoid lineages were detected including NK cells (characterized by high expression of Ccl5), ILC subset 2 (Il7r and Rora), T cells (Trbc2) and B cells (Cd19) (FIG. 1C), while granulocytes and myeloid cells separated into neutrophils (Retnlg), basophils (Mcpt8), mast cells (Mcpt4), DCs (Siglech), monocytes (F13a1) and three different subsets of macrophages (Macrophage I-III; Ear2). Annotation by gene expression was further supported by conventional FACS indices (not shown). Despite its vast heterogeneity, clustering of the none-immune compartment (CD45⁻) revealed the three major lineages, epithelium (marked by Epcam expression), endothelium (Cdh5) and fibroblasts (Colla2). In concordance with previous characterizations of lung development (Treutlein et al., 2014), epithelial cells were separated into epithelium progenitors (high Epcam), AT1 cells (Akap5), AT2 cells (Lamp3), Club cells (Scgb3a2) and ciliated cells (Foxj1) subpopulations, while fibroblast subsets included fibroblast progenitors, smooth muscle cells (Enpp2), matrix fibroblasts (Mfap4) and pericytes (Gucyla3) (FIGS. 1B-C). Overall, these data provide a detailed map of both the abundant and extremely rare lung cell types (>0.1% of all cells) during important periods of development, which can be further used to study the differentiation, maturation and cellular dynamics of the lung.

### Example 2

Lung Compartmentalization is Shaped by Waves of Cellular Dynamics

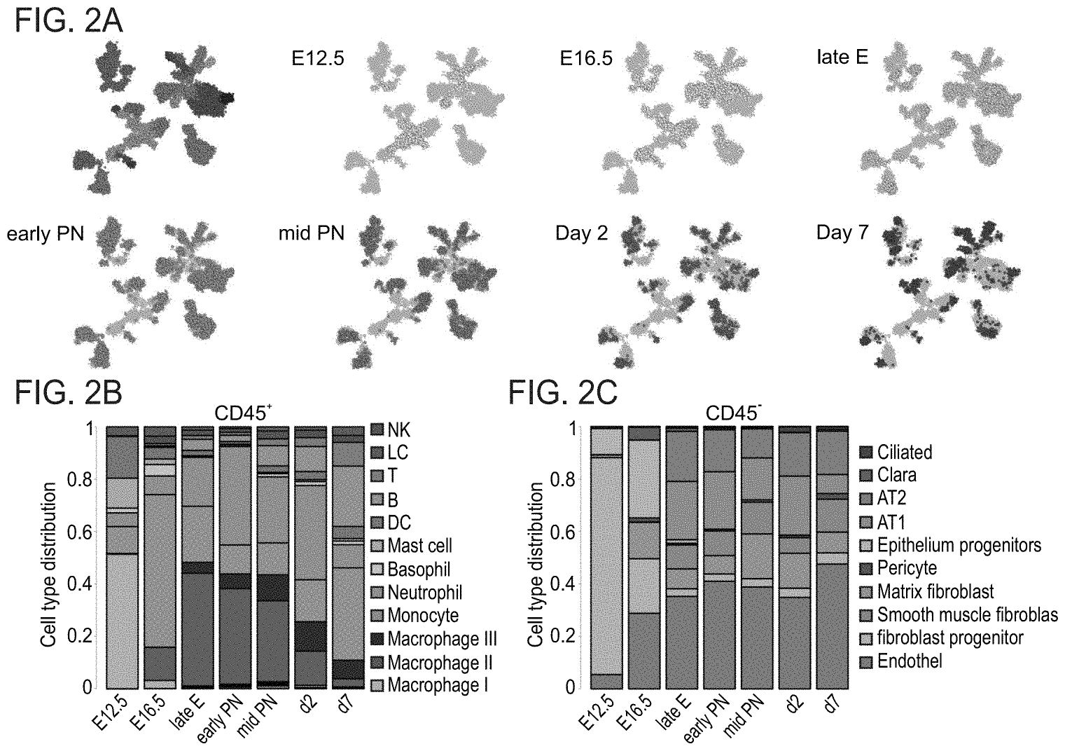

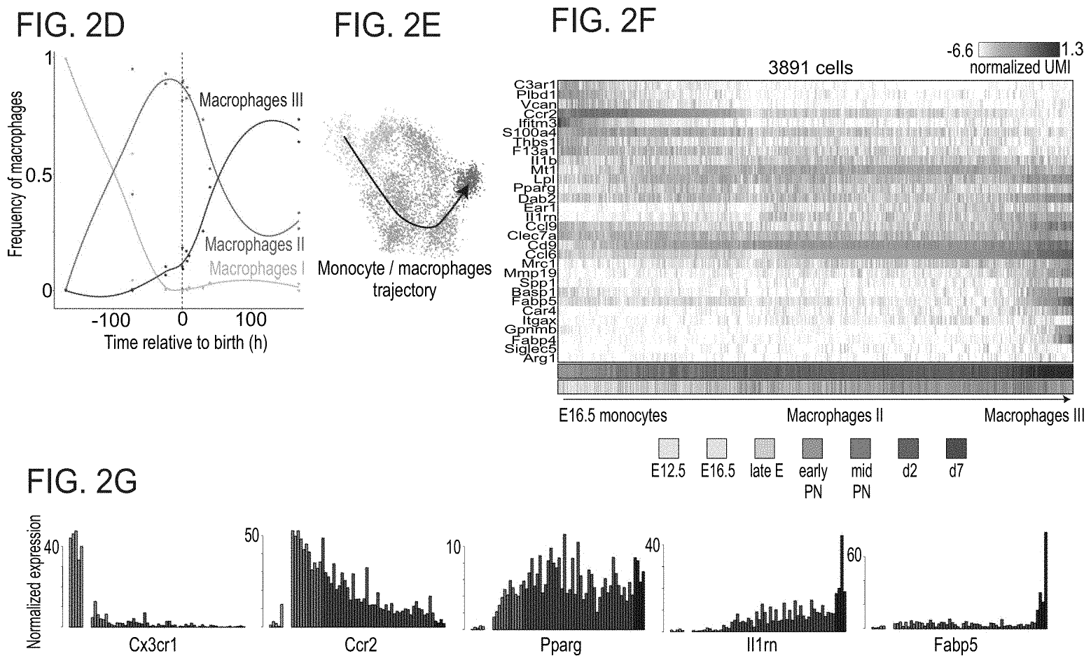

During embryogenesis and soon after birth, the lung undergoes dramatic environmental changes with its maturation and abrupt exposure to airborne oxygen. Accordingly, our analysis shows that meta-cell composition varies widely at these time points (FIG. 2A). At the cell type level, the most prominent cellular dynamics in the immune and non-immune compositions were observed during pregnancy (FIGS. 2B-C). Notably, since tissue dissociation protocols might affect cell type abundances, they can only be regarded as relative quantities (not shown). At the earliest time point (E12.5), the immune compartment was composed mainly of macrophages (51% of CD45⁺ cells), specifically related to subset I, monocytes (10%) and mast cells (11%), whereas at the canalicular stage (E16.5) monocytes, macrophages (subset II), neutrophils and basophils were dominant (58%, 13%, 7% and 4% respectively) and the macrophage I subset was almost diminished. Starting from late pregnancy, all major immune cell populations were present, and later dynamics showed a steady increase in the lymphoid cell compartment (B and T cells), which reached up to 32% of the immune population on day 7 PN, and changes in the composition of the macrophage population (FIG. 2B). Similar to the immune compartment, dynamics in non-immune cell composition were most pronounced during pregnancy (FIG. 2C); E12.5 was composed mainly of undifferentiated fibroblasts (83%) and progenitor epithelial cells (10%). At E16.5, the progenitor epithelial subset continued to increase (30%) and new epithelial cell subsets of club cells (5%) appeared, in parallel to the appearance of pericytes, an increase in endothelium and the appearance of matrix fibroblasts. The cellular composition stabilized from late pregnancy onward, with the appearance of smooth muscle fibroblasts and branching of epithelium into AT1 and AT2 cells (FIG. 2C). These cellular dynamics were consistent across biological replicates (not shown).

In accordance with previous works (Kopf et al., 2015; Tan and Krasnow, 2016), we identified three distinct macrophage subsets, which we term macrophage I-III. These subsets appeared in waves during development, with macrophage I dominating in early pregnancy, macrophage II culminating around birth, and macrophage III steadily increasing since late pregnancy stage, and becoming the majority on day 7 PN (FIG. 2D). Macrophage I cells are transcriptionally distinct from macrophage subsets II-III. Notably, macrophage subsets II-III form a continuous transcriptional spectrum with E16.5 monocytes (FIG. 2E), suggesting that macrophages II and III differentiate from fetal liver monocytes, rather than from macrophage subset I, which might have a yolk sac origin (Ginhoux, 2014; Tan and Krasnow, 2016) (FIG. 2E). To infer the most probable differentiation trajectory for monocytes and macrophage subsets we used Slingshot, for pseudo-time inference (Street et al., 2017), and characterized a gradual acquisition of macrophage genes from E18.5 onward (late E, FIG. 2F). Slingshot trajectory suggests a linear transition of macrophage subsets along the developmental time points. Transcriptionally, macrophage I cells expressed high levels of Cx3cr1 and complement genes (Clqa, Clqb) (FIG. 2G). Macrophage II were molecularly reminiscent of monocytes, expressing Ccr2, F13a1 and Il1b, and intermediate levels of alveolar macrophage (AM)-hallmark genes, such as Him, Lpl, Pparg and Clec7a (Kopf et al., 2015; Schneider et al., 2014) (FIG. 2G). Macrophage III expressed a unique set of AM hallmark genes, including; Pparg, Fabp4, Fabp5, Il1m, Car4, Lpl, Clec7a and Itgax (Gautier et al., 2012; Lavin et al., 2014) (FIGS. 2F-G). We similarly reconstructed the differentiation waves in the fibroblast and epithelial lineages, highlighting the main genes associated with the branching of smooth muscle and matrix fibroblasts (not shown), and priming of epithelium progenitors into AT1 and AT2 cells (not shown). Together, our data reveal tightly regulated dynamic changes in both cell type composition and gene expression programs along lung development. These cellular and molecular dynamics across different cell types suggest that these programs are orchestrated by a complex network of cellular crosstalk.

### Example 3

Lung basophils broadly interact with the immune and non-immune compartments In multicellular organisms, tissue function emerges as heterogeneous cell types form complex communication networks, which are mediated primarily by interactions between ligands and receptors (LR) (Zhou et al., 2018). Examining LR pairs in single cell maps can potentially reveal central cellular components shaping tissue fate (Camp et al., 2017; Zhou et al., 2018). In order to systematically map cellular interactions between cells and reveal potential communication factors controlling development, we characterized LR pairs between all lung cell types (FIG. 3A). Briefly, we filtered all LR expressed in at least one meta-cell and associated each ligand or receptor with its expression profile across all cells and along the developmental time points, using a published dataset linking ligands to their receptors (Methods) (Ramilowski et al., 2015).

In the developing lung, modules of LR mainly clustered by cell type (not shown). However, for some LR we could identify significant changes in expression levels in the same cell type during development (not shown). We projected ligands and receptors based on their correlation structure, resulting in a graphical representation of all LR and their interactions, which highlighted their separation into cell type related modules (FIG. 3B, Methods). The lung LR map showed a clear separation between the communication patterns of the immune and non-immune compartments (FIG. 3C), characterized by enrichment of LR interactions between the immune compartment (I) and itself and between the non-immune compartment (NI) and itself, and depletion of interactions between compartments (I-I and NI-NI interactions, p<10⁻⁴, not shown). Notably, whereas the majority of crosstalk occurs within each compartment, sporadic I-NI and NI-I interactions might include key signaling pathways for tissue development and homeostasis. We next classified specific ligand families and pathways into functional groups

(Methods). As expected, cytokines and components of the complement system were found mainly in the immune compartment, as well as the receptors recognizing them (FIGS. 3D-E). Complementarily, the non-immune compartment was enriched for growth factors, matrix signaling and cell adhesion ligands and receptors (FIGS. 3D-E).

To identify important cellular communication hubs involved in a large number of interactions between and within compartments, we examined LR expression patterns across different cell types (not shown). From the non-immune compartment, smooth muscle fibroblasts, expressing Tgfb3 and the Wnt ligand Wnt5a (Nabhan et al., 2018), and AT2 cells, characterized by the exclusive expression of interleukin 33 (Il33) and surfactant protein (Sfpta1), were involved in complex NI-NI and NI-I signaling (FIGS. 3F-G) (Saluzzo et al., 2017). Within the immune compartment, we observed expression of hallmark receptors important for differentiation and maturation of unique cell subsets, such as Csf2rb and Csf1r in monocytes and macrophages (Ginhoux, 2014; Guilliams et al., 2013; Schneider et al., 2014) (not shown). ILC, previously implicated to play an important role in the differentiation of AM (de Kleer et al., 2016; Saluzzo et al., 2017), were found here as the major cells expressing Csf2 (GM-CSF, FIG. 3H). Surprisingly, basophils, comprising a rare population of the immune compartment (1.5%), displayed a rich and complex LR profile, interacting with both the immune and the non-immune compartments. The interaction map highlighted basophils as the main source of many key cytokines and growth factors, such as Csf1, Il6, Il13 and Hgf (FIG. 3I), and their counterpart receptors were expressed by unique resident lung cells. Overall, our analysis confirms important and established LR interactions in the process of lung development, while discovering potential novel crosstalk circuits between and within lung immune and non-immune cell types.

### Example 4

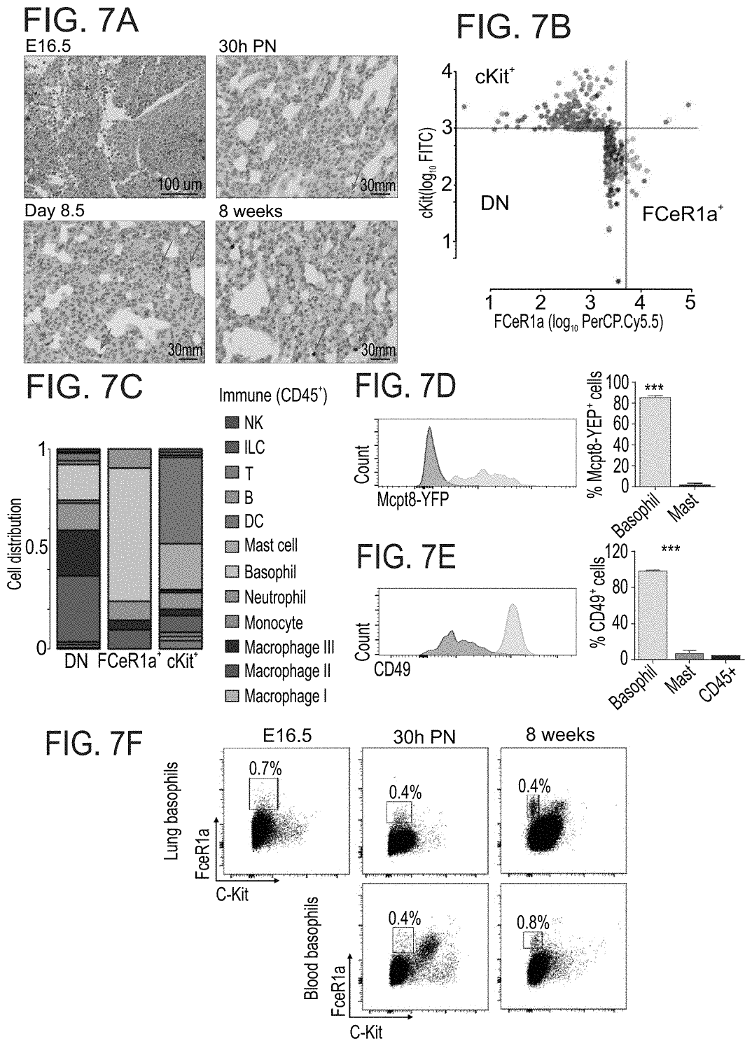

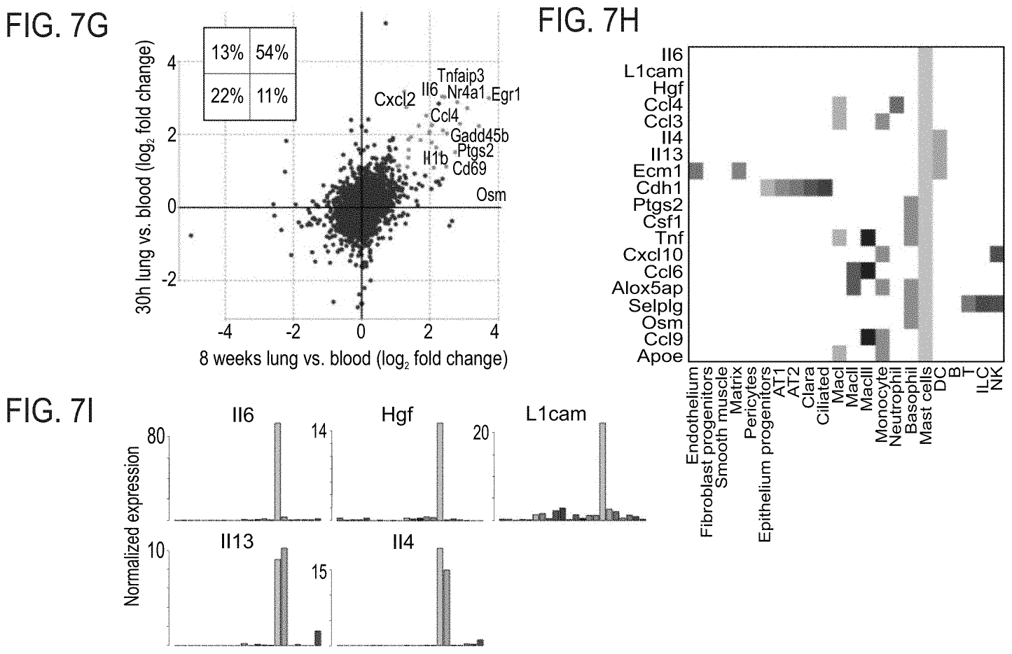

Lung basophils are characterized by distinct spatial localization and gene signature In light of the rich interactive profile of basophils (FIG. 3I), we hypothesized that these cells may have a central role in cellular communication within the lung, both by responding to lung cues and by modifying the microenvironment. In order to identify the spatial localization of lung basophils, we implemented a Mcpt8^{YFP/+} transgenic mouse model, and observed that YFP⁺ basophils within the lung parenchyma were localized in close proximity to alveoli at 30h PN, on day 8.5 PN and in 8 weeks old mice (FIG. 7A). We combined TissueFAXS images of whole lobe sections together with a semi-automated computational analysis to accurately identify basophils and quantify their spatial localization in the lung (Methods). We observed that basophils were more likely to reside in proximity to alveoli than randomly selected cells, on day 8.5 PN, and to a lesser extent, in 8 weeks old adult mice (FIGS. 4A-B, Methods). In order to further measure basophil spatial organization in the lung parenchyma, we performed tissue clearing followed by left lung lobe imagining of Mcpt8^{YFP/+} mice at different time points. Anti-GFP antibody staining further confirmed that basophils were distributed all over the lung lobes (FIG. 4C).

To molecularly characterize lung basophils, we sought to extensively isolate them by flow cytometry. We gated on basophil specific markers identified in the data (CD45⁺FceR1α⁺cKit⁻), and validated our sorting strategy using MARS-seq analysis (FIGS. 7B-C). Analysis of Mcpt8^{YFP/+} transgenic mice showed that 84% of CD45⁺FceR1α⁺cKit⁻cells are YFP⁺ cells, and that 98% express the basophil marker CD49b (FIGS. 7D-E). Basophil quantification per whole lung tissue showed a gradual accumulation of this population along tissue development (FIG. 4D), and its percentage within the immune population (CD45⁺) remained stable (FIG. 7F). To inspect whether lung basophils have a unique resident expression program that is not observed in the circulation, we sorted time point matched basophils from lung and peripheral blood for MARS-seq analysis (FIG. 7F). The gene expression profile of lung basophils differed from blood-circulating basophils, characterized by a unique gene signature, that includes expression of Il6, Il13, Cxcl2, Tnf, Osm and Ccl4 (FIGS. 4E-F). This unique gene signature persisted in the adult lung resident basophils (FIGS. 4F-G, 7G, Table 4).

TABLE 4

| | E16.5X-lung | PN_30hX-lung | PN_8wX-lung |
|---|---|---|---|
| Alox5ap | 561.644989 | 568.008484 | 363.025263 |
| Apoe | 541.385795 | 229.071735 | 43.2550202 |
| Ccl3 | 689.601793 | 1427.84118 | 1787.19771 |
| Ccl4 | 286.807732 | 756.732619 | 1247.64324 |
| Ccl6 | 541.816111 | 927.645066 | 1625.5779 |
| Ccl9 | 793.921455 | 798.87764 | 734.381808 |
| Cdh1 | 96.6176845 | 97.6629511 | 93.4221015 |
| Csf1 | 91.6689123 | 170.891149 | 384.46173 |
| Cxcl10 | 0 | 0 | 3.02568054 |
| Ecm1 | 63.6756189 | 20.1583453 | 4.08139808 |
| Hdc | 337.024663 | 637.071119 | 520.369883 |
| Il13 | 22.5662839 | 37.1237111 | 12.5166021 |
| Il4 | 31.6511834 | 65.0012354 | 40.5774162 |
| Il6 | 164.851204 | 477.953659 | 454.718712 |
| L1cam | 38.2886224 | 65.1045739 | 52.3608046 |
| Osm | 113.059078 | 294.204465 | 294.993523 |
| Ptgs2 | 26.3848596 | 56.413578 | 71.4765609 |
| Selplg | 131.305367 | 130.060808 | 183.129092E19 |
| Tnf | 56.488424 | 61.3531137 | 15.5495109 |
| Vasp | 107.14211 | 114.61843 | 99.5063923 |
| Alox5 | 83.1435448 | 66.0501975 | 41.1434429 |
| C3ar1 | 72.3719322 | 63.7697019 | 98.5028154 |
| Ccr2 | 193.314909 | 155.904043 | 50.5823799 |
| Cd53 | 130.624049 | 123.974346 | 126.411257 |
| Cd63 | 124.667977 | 131.818839 | 142.672453 |
| Csf2rb | 267.360255 | 407.511692 | 383.995605 |
| Cxcr4 | 26.6428388 | 30.1027335 | 161.238197 |
| Fcer1a | 87.1551924 | 67.6920649 | 200.763458 |
| Fgfr1 | 20.8731568 | 21.0484723 | 2.31295623 |
| Gpr56 | 39.9549815 | 14.9398622 | 27.9669149 |
| Ifitm1 | 1702.73081 | 1396.34002 | 635.889343 |
| Il18rap | 90.8662942 | 170.644028 | 159.195304 |
| Il1rl1 | 166.031029 | 105.590709 | 30.6365344 |
| Il2ra | 23.5522382 | 7.01675782 | 0.18136633 |
| Il7r | 24.849006 | 17.9011009 | 39.5680045 |
| Itgam | 77.1569795 | 93.2379187 | 47.1554709 |
| Itgb7 | 108.840347 | 88.5164853 | 78.069971 |
| P2ry14 | 65.0260974 | 50.3833595 | 32.0291284 |
| Sell | 41.898773 | 55.5469404 | 71.1873557 |
| Slc18a2 | 31.3824811 | 19.9113696 | 55.6472694 |
| Tyrobp | 599.392106 | 519.965051 | 574.517551 |

Notably, the ligands Il6, Hgf and L1cam are exclusively expressed by lung basophils, compared to other lung immune and non-immune cells (FIGS. 7H-I). Together, we show that lung resident basophils reside within the tissue parenchyma, specifically localize near the alveoli, and acquire distinct and persistent lung- characteristic signaling and gene program compared to their circulating counterparts.

### Example 5

IL33 and GM-CSF Imprint the Lung-Alveolar Basophil Transcriptional Identity

Since lung-resident basophils showed a unique gene expression signature, we analyzed the data for lung specific signals that can serve as differentiation cues for lung basophil receptors (not shown). Csf2 (GM-CSF) is a hematopoietic growth factor, whose role in shaping the lung microenvironment and specifically AM, has long been established (Ginhoux, 2014; Guilliams et al., 2013; Shibata et al., 2001). Interestingly, we found that the major source of Csf2 expression in the lung stemmed from ILC and the basophils themselves, with only a small contribution from AT2 cells. Among all lung cells, basophils expressed the highest RNA and protein levels of Csf2rb, a major receptor for Csf2 (FIGS. 5A-B). In addition, basophils and mast cells expressed the highest RNA and protein levels of the receptor Il1rl1 (IL33R/ST2), which specifically binds Il33 (FIGS. 5C-D). Previous reports identified IL-33 as a major driver for cellular differentiation and lung maturation, expressed mainly by AT2 cells. Specifically, lung ILC-2 were previously reported to depend on IL33-ST2 signaling for their function (de Kleer et al., 2016; Saluzzo et al., 2017). Single-molecule fluorescent in-situ hybridization (smFISH) staining of post-natal lung tissue for Il1rl1 and 1133 genes, together with the basophil marker Mcpt8, showed co-expression of these genes in neighboring cells, suggesting that basophils and AT2 cells reside in spatial proximity in the lung tissue (FIG. 5E). Immunohistochemistry (IHC) staining of AT2 and basophils at adult lung tissue further confirmed these results and localized this signaling in the alveoli niche (FIG. 5F). To functionally validate the effects of IL-33 signaling on the lung-basophil gene expression profile, we purified basophils from the lungs of Il1rl1 (IL33R) knockout mice for MARS-seq analysis. We found that Il1rl1 deficient lung basophils lacked expression of many of the genes specific to lung-resident basophils, and showed higher similarity to blood circulating basophils (FIGS. 5G-H, 8A), suggesting that IL-33 signaling is mediating a large part of the specific gene signature of lung basophils.

In order to test whether the lung environmental signals, IL-33 and GM-CSF, are directly responsible for inducing the lung basophil phenotype, we used an in vitro system where we cultured bone marrow (BM)-derived basophils in media supplemented with these cytokines. We differentiated BM-derived cells in IL3 supplemented medium, isolated basophils by negative selection of cKit (BM-basophils), and cultured them in the presence of growth media alone (IL3) or with different combinations of the lung cytokine milieu; GM-CSF and/or IL-33 (FIGS. 5I, 8B-C). We found that IL-33 and GM-CSF each induced a specific transcriptional program (FIG. 8D). IL-33 induced a major part of the lung basophil gene signature including the ligands Il6, Il13, Il1b, Tnf, Cxcl2 and Csf2, as well as the transcription factor Pou2f2 (FIGS. 5J, 8E), while GM-CSF induced a smaller set of the lung basophil gene program. Interestingly, we found that cells cultured with both GM-CSF and IL-33 activated both programs, suggesting a combinatorial effect of both cytokines on the BM-basophil signature (FIGS. 5K, 8F). Furthermore, revisiting the in vivo lung and blood basophils by projecting their gene expression profile on the GM-CSF/IL-33 differentiation programs, revealed a time-point independent up-regulation of both expression programs in lung-resident basophils compared to basophils from circulation (FIG. 5L). Further support for two independent signaling programs was derived from the Il1rl1 knockout mice, which showed that II1rl1-knockout basophils perturbed the IL-33 program without any change in expression of GM-CSF induced genes (FIG. 8G). Together, a combination of knockout data and in vitro assay demonstrate that the lung environment imprints a robust transcriptional program in basophils, which is mediated by at least two independent signaling pathways, dominated by IL-33 and with minor contribution of GM-CSF.

### Example 6

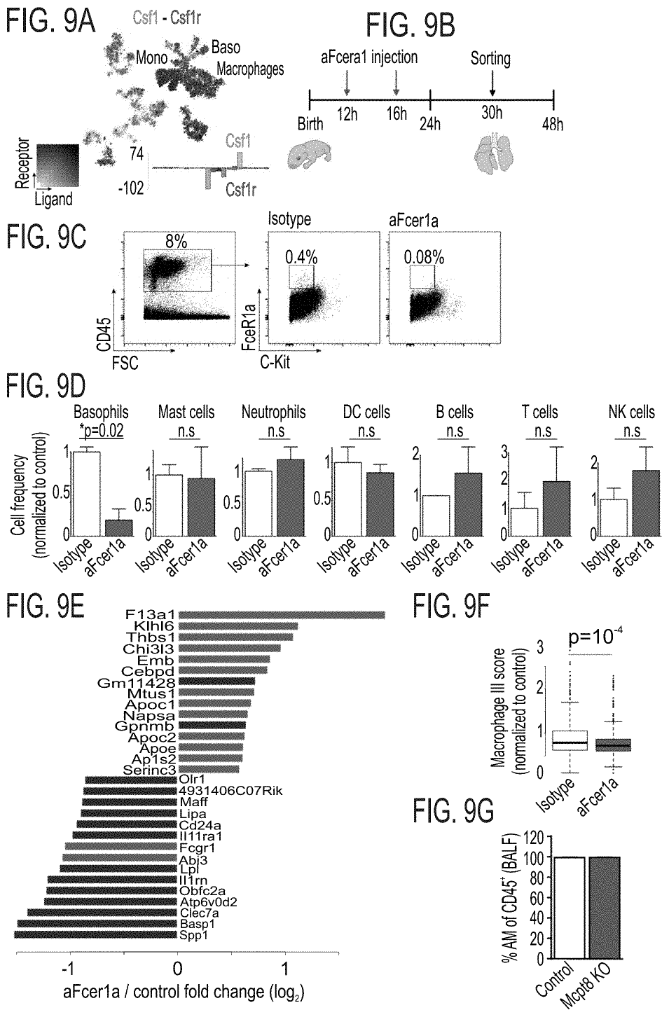

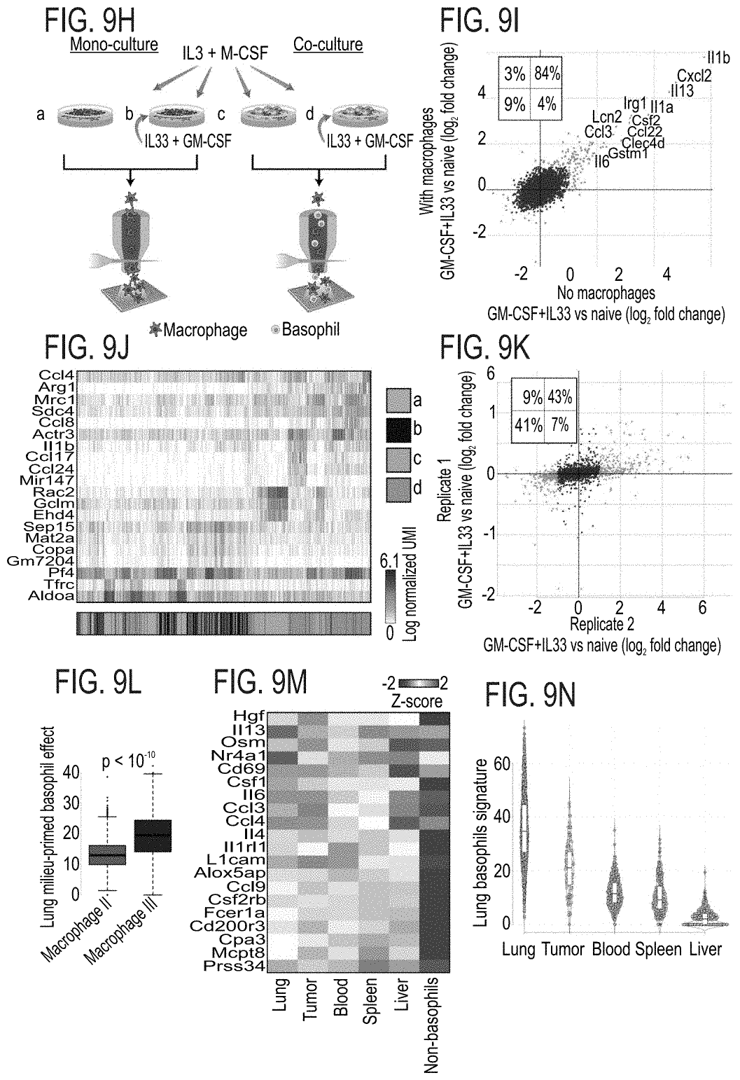

Lung basophils imprint naive macrophages with an alveolar macrophage phenotype The expression of critical lung signaling molecules by basophils prompted us to explore their signaling activity, and contribution in shaping the unique phenotype of other lung resident cells. As lung resident basophils highly express Il6, Il13 and Csfl, three important myeloid growth factors, we hypothesized that they may interact with other myeloid cells, particularly macrophages, via Il6ra, Il13ra and Csf1r (FIGS. 3A-I, 6A-D, 9A). IHC of basophils (Mcpt8) and macrophages (F4/80) showed their spatial proximity within lung parenchyma during the alveolarization process (FIG. 6E). In order to evaluate the impact of basophils on macrophage differentiation, we tested the effect of lung-basophil depletion on the maturation of lung myeloid cells. For this purpose, we administered anti-Fcεr1α (MARI) antibody or isotype control intra-nasally to neonatal mice to induce local depletion of basophils (two injections at 12h and 16h PN; Methods), and collected lung CD45⁺ cells 30h PN for MARS-seq analysis (FIG. 9B). The anti-Fcεr1α antibody efficiently and specifically depleted basophils in the lung, without perturbing the frequencies of other immune cells, determined both by FACS and MARS-seq (FIGS. 6F, 9C-D). Lung basophil depletion was coupled with a reduction of the AM fraction (Macrophage III) within the macrophage compartment (FIG. 6G). Moreover, macrophages derived from basophil-depleted lungs showed a decrease in expression of genes reminiscent of mature AM, including an anti-inflammatory (M2) module (Clec7a, Ccl17), and an increase in genes related to macrophage II (p=10⁻⁴; FIGS. 6H, 9E-F). Specifically, we observed down regulation in the levels of Il1rn, Ear1, Lpl, Clec7a and Siglec5, hallmark genes of AM, concomitantly with the induction of F13a1, a gene shared by macrophage II and monocytes (FIG. 6I). Since a proper AM maturation process is critical for their role in lung-immunomodulation and as phagocytic cells, we further characterized the effect of constitutive basophil depletion on AM function in adults. For this, we compared cells derived from bronchoalveolar lavage fluid (BALF) of adult Mcpt8^{cre/+} DTA^{fl/+} mice, depleted specifically of basophils, to littermate controls. In both conditions, BALF cells consisted of 98% AM (FIG. 9G). However, Mcpt8^{cre/+} DTA^{fl/+} BALF had an overall lower cell count compared to control littermates (FIG. 6J). Importantly, Mcpt8^{cre/+} DTA^{fl/+} derived AM were impaired in the phagocytosis of inactivated bacteria compared to controls (FIG. 6K). Together, our data show that the lung-basophil AM niche is important for differentiation, compartmentalization and phagocytic properties of AM.

The effect of lung basophils on AM maturation in vivo, led us to ask whether lung-basophils can promote transition of monocytes or naïve macrophages towards the AM signature directly. For this hypothesis, we performed an in vitro co-culturing assay. Naïve BM-derived macrophages (BM-MΦ) were cultured alone or co-cultured with BM-basophils in growth media supporting both cell types (M-CSF and IL-3, respectively), with or without a combination of

GM-CSF and IL-33, the milieu signaling that primes basophils toward the lung-basophil phenotype (FIG. 9H, Methods). Co-culturing of BM-basophils with BM-MΦ did not change the previously characterized basophil phenotype in any condition (FIG. 91). However, meta-cell analysis showed a clear distinction between BM-MΦ that were cultured with and without basophils (FIG. 6L). Importantly, only BM-MΦ grown in the presence of lung milieu-primed (GM-CSF+IL33) basophils upregulated genes associated to AM, including an anti-inflammatory (M2) module (Cc17, Clec7a, Arg1 and Itgax; FIGS. 6L-M, 9J). Notably, this effect on BM-MΦ polarization was not seen when macrophages were cultured in a medium that was supplemented with lung environmental cytokines (GM-CSF and IL-33) alone, showing that these cytokines mediate the signaling effect via basophils (FIGS. 6L-M). We characterized a large and reproducible change in gene expression of BM-MΦ co-cultured with lung milieu-primed basophils compared to all other conditions, affecting many genes differentially expressed between macrophage subsets III (mature AM) and II, previously associated with the alternative anti-inflammatory (M2) polarization phenotype (p<10⁻¹⁰; FIGS. 6M-N, 9K-L) (Gordon, 2003). To further examine the direct effect of lung milieu-primed basophils on AM maturation, we next purified CD45⁺CD115⁺ myeloid cells containing mainly monocytes and undifferentiated AM from 30h PN lungs, and performed the co-culture experiment (FIG. 9G). Importantly, the same lung basophil program induced in naïve BM-MΦ in vitro (FIG. 6M), was also up-regulated in monocytes and undifferentiated AM that were cultured with lung milieu-primed basophils (GM-CSF+IL-33) (FIG. 60), while it was down-regulated in macrophages derived from basophil depleted lungs (FIG. 6P). These data suggest that the basophil phenotype might be imprinted by tissue environmental cues, and as a result, they mediate immunomodulating activities in tissue myeloid cells. We therefore compared gene expression profiles of basophils derived from lungs of 8-week old mice to basophils isolated from the tumor microenvironment of B16 melanoma cell line injected mice, and from spleen and liver of 8 weeks old mice (FIG. 9M). While all tissue basophils highly expressed basophil marker genes (e.g. Mcpt8, Cpa3, Cd200r3, Fcer1α), the lung signature was exclusive, with higher similarity to tumor-derived basophils, mainly in expression of immune suppression genes, such as Il4, Il6, Osm and Il13 (FIGS. 9M-N). Taken together, our data indicate that the instructive signals from the lung environment imprint basophils with a unique signature of cytokines and growth factors, which subsequently propagate important signals to other lung resident cells, including the polarization of AM towards phagocytic and anti-inflammatory macrophages.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited throughout the Application

Achim, K., Pettit, J. B., Saraiva, L. R., Gavriouchkina, D., Larsson, T., Arendt, D., and Marioni, J. C. (2015). High-throughput spatial mapping of single-cell RNA-seq data to tissue of origin. Nat Biotechnol 33, 503-509.

Ayaub, E. A., Dubey, A., Imani, J., Botelho, F., Kolb, M. R. J., Richards, C. D., and Ask, K. (2017). Overexpression of OSM and IL-6 impacts the polarization of pro-fibrotic macrophages and the development of bleomycin-induced lung fibrosis. Sci Rep 7, 13281.

Bendall, S. C., Davis, K. L., Amir el, A.D., Tadmor, M. D., Simonds, E. F., Chen, T. J., Shenfeld, D. K., Nolan, G. P., and Pe'er, D. (2014). Single-cell trajectory detection uncovers progression and regulatory coordination in human B cell development. Cell 157, 714-725.

Butovsky, O., Jedrychowski, M. P., Moore, C. S., Cialic, R., Lanser, A. J., Gabriely, G., Koeglsperger, T., Dake, B., Wu, P. M., Doykan, C. E., et al. (2014). Identification of a unique TGF-beta-dependent molecular and functional signature in microglia. Nat Neurosci 17, 131-143.

Camp, J. G., Sekine, K., Gerber, T., Loeffler-Wirth, H., Binder, H., Gac, M., Kanton, S., Kageyama, J., Damm, G., Seehofer, D., et al. (2017). Multilineage communication regulates human liver bud development from pluripotency. Nature 546, 533-538.

Camp, J. G., and Treutlein, B. (2017). Human organomics: a fresh approach to understanding human development using single-cell transcriptomics. Development 144, 1584-1587.

Cardoso, W. V., and Lu, J. (2006). Regulation of early lung morphogenesis: questions, facts and controversies. Development 133, 1611-1624.

Chhiba, K. D., Hsu, C. L., Berdnikovs, S., and Bryce, P.J. (2017). Transcriptional Heterogeneity of Mast Cells and Basophils upon Activation. J Immunol 198, 4868-4878.

Cipolletta, D., Cohen, P., Spiegelman, B. M., Benoist, C., and Mathis, D. (2015). Appearance and disappearance of the mRNA signature characteristic of Treg cells in visceral adipose tissue: age, diet, and PPARgamma effects. Proc Natl Acad Sci U S A 112, 482-487.

Cohen, M., Ben-Yehuda, H., Porat, Z., Raposo, C., Gordon, S., and Schwartz, M. (2017). Newly Formed Endothelial Cells Regulate Myeloid Cell Activity Following Spinal Cord Injury via Expression of CD200 Ligand. J Neurosci 37, 972-985.

Cohen, M., Matcovitch, O., David, E., Barnett-Itzhaki, Z., Keren-Shaul, H., Blecher-Gonen, R., Jaitin, D. A., Sica, A., Amit, I., and Schwartz, M. (2014). Chronic exposure to TGFbetal regulates myeloid cell inflammatory response in an IRF7-dependent manner. EMBO J 33, 2906-2921.

Cordeiro Gomes, A., Hara, T., Lim, V. Y., Herndler-Brandstetter, D., Nevius, E., Sugiyama, T., Tani-Ichi, S., Schlenner, S., Richie, E., Rodewald, H. R., et al. (2016). Hematopoietic Stem Cell Niches Produce Lineage-Instructive Signals to Control Multipotent Progenitor Differentiation. Immunity 45, 1219-1231.

de Kleer, I. M., Kool, M., de Bruijn, M. J., Willart, M., van Moorleghem, J., Schuijs, M. J., Plantinga, M., Beyaert, R., Hams, E., Fallon, P. G., et al. (2016). Perinatal Activation of the Interleukin-33 Pathway Promotes Type 2 Immunity in the Developing Lung. Immunity 45, 1285-1298.

Denzel, A., Maus, U.A., Rodriguez Gomez, M., Moll, C., Niedermeier, M., Winter, C., Maus, R., Hollingshead, S., Briles, D. E., Kunz-Schughart, L.A., et al. (2008). Basophils enhance immunological memory responses. Nat Immunol 9, 733-742.

Desai, T.J., Brownfield, D. G., and Krasnow, M. A. (2014). Alveolar progenitor and stem cells in lung development, renewal and cancer. Nature 507, 190-194.

Donati, G. (2016). The niche in single-cell technologies. Immunol Cell Biol 94, 250-255.

Drost, J., Artegiani, B., and Clevers, H. (2016). The Generation of Organoids for Studying Wnt Signaling. Methods Mol Biol 1481, 141-159.

Epelman, S., Lavine, K. J., Beaudin, A. E., Sojka, D. K., Carrero, J. A., Calderon, B., Brija, T., Gautier, E. L., Ivanov, S., Satpathy, A. T., et al. (2014). Embryonic and adult-derived resident cardiac macrophages are maintained through distinct mechanisms at steady state and during inflammation. Immunity 40, 91-104.

Fejer, G., Wegner, M. D., Gyory, I., Cohen, I., Engelhard, P., Voronov, E., Manke, T., Ruzsics, Z., Dolken, L., Prazeres da Costa, O., et al. (2013). Nontransformed, GM-CSF-dependent macrophage lines are a unique model to study tissue macrophage functions. Proc Natl Acad Sci U S A 110, E2191-2198.

Galli, S. J., Gordon, J. R., and Wershil, B. K. (1991). Cytokine production by mast cells and basophils. Curr Opin Immunol 3, 865-872.

Gautier, E. L., Shay, T., Miller, J., Greter, M., Jakubzick, C., Ivanov, S., Helft, J., Chow, A., Elpek, K. G., Gordonov, S., et al. (2012). Gene-expression profiles and transcriptional regulatory pathways that underlie the identity and diversity of mouse tissue macrophages. Nat Immunol /3, 1118-1128.

Giladi, A., and Amit, I. (2018). Single-Cell Genomics: A Stepping Stone for Future Immunology Discoveries. Cell 1 72, 14-21.

Ginhoux, F. (2014). Fate PPAR-titioning: PPAR-gamma ‘instructs’ alveolar macrophage development. Nat Immunol 15, 1005-1007.

Ginhoux, F., Greter, M., Leboeuf, M., Nandi, S., See, P., Gokhan, S., Mehler, M. F., Conway, S. J., Ng, L. G., Stanley, E. R., et al. (2010). Fate mapping analysis reveals that adult microglia derive from primitive macrophages. Science 330, 841-845.

Ginhoux, F., and Jung, S. (2014). Monocytes and macrophages: developmental pathways and tissue homeostasis. Nat Rev Immunol 14, 392-404.

Gjorevski, N., Sachs, N., Manfrin, A., Giger, S., Bragina, M. E., Ordonez-Moran, P., Clevers, H., and Lutolf, M. P. (2016). Designer matrices for intestinal stem cell and organoid culture. Nature 539, 560-564.

Gordon, S. (2003). Alternative activation of macrophages. Nat Rev Immunol 3, 23-35. Gordon, S., and Taylor, P. R. (2005). Monocyte and macrophage heterogeneity. Nat Rev Immunol 5, 953-964.

Gosselin, D., Link, V. M., Romanoski, C. E., Fonseca, G. J., Eichenfield, D. Z., Spann, N. J., Stender, J. D., Chun, H. B., Garner, H., Geissmann, F., et al. (2014). Environment drives selection and function of enhancers controlling tissue-specific macrophage identities. Cell 159, 1327-1340.

Greter, M., Helft, J., Chow, A., Hashimoto, D., Mortha, A., Agudo-Cantero, J., Bogunovic, M., Gautier, E. L., Miller, J., Leboeuf, M., et al. (2012). GM-CSF controls nonlymphoid tissue dendritic cell homeostasis but is dispensable for the differentiation of inflammatory dendritic cells. Immunity 36, 1031-1046.

Grun, D., Lyubimova, A., Kester, L., Wiebrands, K., Basak, O., Sasaki, N., Clevers, H., and van Oudenaarden, A. (2015). Single-cell messenger RNA sequencing reveals rare intestinal cell types. Nature 525, 251-255.

Guilliams, M., De Kleer, I., Henri, S., Post, S., Vanhoutte, L., De Prijck, S., Deswarte, K., Malissen, B., Hammad, H., and Lambrecht, B. N. (2013a). Alveolar macrophages develop from fetal monocytes that differentiate into long-lived cells in the first week of life via GM-CSF. J Exp Med 210, 1977-1992.

Guilliams, M., Lambrecht, B. N., and Hammad, H. (2013b). Division of labor between lung dendritic cells and macrophages in the defense against pulmonary infections. Mucosal Immunol 6, 464-473.

Gury-BenAri, M., Thaiss, C. A., Serafini, N., Winter, D. R., Giladi, A., Lara-Astiaso, D., Levy, M., Salame, T. M., Weiner, A., David, E., et al. (2016). The Spectrum and Regulatory Landscape of Intestinal Innate Lymphoid Cells Are Shaped by the Microbiome. Cell 166, 1231-1246 e1213.

Haber, A. L., Biton, M., Rogel, N., Herbst, R. H., Shekhar, K., Smillie, C., Burgin, G., Delorey, T. M., Howitt, M. R., Katz, Y., et al. (2017). A single-cell survey of the small intestinal epithelium. Nature 551, 333-339.

Halpern, K. B., Shenhav, R., Matcovitch-Natan, O., Toth, B., Lemze, D., Golan, M., Massasa, E. E., Baydatch, S., Landen, S., Moor, A. E., et al. (2017). Single-cell spatial reconstruction reveals global division of labour in the mammalian liver. Nature 542, 352-356.

Hart, Y., Reich-Zeliger, S., Antebi, Y. E., Zaretsky, I., Mayo, A. E., Alon, U., and Friedman, N. (2014). Paradoxical signaling by a secreted molecule leads to homeostasis of cell levels. Cell 158, 1022-1032.

Hashimoto, D., Chow, A., Noizat, C., Teo, P., Beasley, M. B., Leboeuf, M., Becker, C. D., See, P., Price, J., Lucas, D., et al. (2013). Tissue-resident macrophages self-maintain locally throughout adult life with minimal contribution from circulating monocytes. Immunity 38, 792-804.

Hoeffel, G., and Ginhoux, F. (2018). Fetal monocytes and the origins of tissue-resident macrophages. Cell Immunol.

Huang da, W., Sherman, B. T., and Lempicki, R. A. (2009a). Bioinformatics enrichment tools: paths toward the comprehensive functional analysis of large gene lists. Nucleic Acids Res 37, 1-13.

Huang da, W., Sherman, B. T., and Lempicki, R. A. (2009b). Systematic and integrative analysis of large gene lists using DAVID bioinformatics resources. Nat Protoc 4, 44-57.

Hussell, T., and Bell, T. J. (2014). Alveolar macrophages: plasticity in a tissue-specific context. Nat Rev Immunol 14, 81-93.

Ibarra-Soria, X., Jawaid, W., Pijuan-Sala, B., Ladopoulos, V., Scialdone, A., Jorg, D. J., Tyser, R. C. V., Calero-Nieto, F. J., Mulas, C., Nichols, J., et al. (2018). Defining murine organogenesis at single-cell resolution reveals a role for the leukotriene pathway in regulating blood progenitor formation. Nat Cell Biol 20, 127-134.

Itzkovitz, S., Blat, I. C., Jacks, T., Clevers, H., and van Oudenaarden, A. (2012). Optimality in the development of intestinal crypts. Cell 148, 608-619.

Jaitin, D. A., Kenigsberg, E., Keren-Shaul, H., Elefant, N., Paul, F., Zaretsky, I., Mildner, A., Cohen, N., Jung, S., Tanay, A., et al. (2014). Massively parallel single-cell RNA-seq for marker-free decomposition of tissues into cell types. Science 343, 776-779.

Jones, C. V., Williams, T. M., Walker, K. A., Dickinson, H., Sakkal, S., Rumballe, B. A., Little, M. H., Jenkin, G., and Ricardo, S. D. (2013). M2 macrophage polarisation is associated with alveolar formation during postnatal lung development. Respir Res 14, 41.

Kageyama, R., Ohtsuka, T., Shimojo, H., and Imayoshi, I. (2008). Dynamic Notch signaling in neural progenitor cells and a revised view of lateral inhibition. Nat Neurosci 11, 1247-1251.

Kawakami, T., and Galli, S. J. (2002). Regulation of mast-cell and basophil function and survival by IgE. Nat Rev Immunol 2, 773-786.

Keren-Shaul, H., Spinrad, A., Weiner, A., Matcovitch-Natan, O., Dvir-Szternfeld, R., Ulland, T. K., David, E., Baruch, K., Lara-Astaiso, D., Toth, B., et al. (2017). A Unique Microglia Type Associated with Restricting Development of Alzheimer's Disease. Cell 169, 1276-1290 e1217.

Kim, D., Langmead, B., and Salzberg, S. L. (2015). HISAT: a fast spliced aligner with low memory requirements. Nat Methods 12, 357-360.

Klein, A. M., and Macosko, E. (2017). InDrops and Drop-seq technologies for single-cell sequencing. Lab Chip 17, 2540-2541.

Klein, A. M., Mazutis, L., Akartuna, I., Tallapragada, N., Veres, A., Li, V., Peshkin, L., Weitz, D. A., and Kirschner, M. W. (2015). Droplet barcoding for single-cell transcriptomics applied to embryonic stem cells. Cell 161, 1187-1201.

Kopf, M., Schneider, C., and Nobs, S. P. (2015). The development and function of lung-resident macrophages and dendritic cells. Nat Immunol 16, 36-44. Kretzschmar, K., and Clevers, H. (2016). Organoids: Modeling Development and the Stem Cell Niche in a Dish. Dev Cell 38, 590-600.

Lavin, Y., Kobayashi, S., Leader, A., Amir, E.D., Elefant, N., Bigenwald, C., Remark, R., Sweeney, R., Becker, C. D., Levine, J. H., et al. (2017). Innate Immune Landscape in Early Lung Adenocarcinoma by Paired Single-Cell Analyses. Cell 169, 750-765 e717.

Lavin, Y., Winter, D., Blecher-Gonen, R., David, E., Keren-Shaul, H., Merad, M., Jung, S., and Amit, I. (2014). Tissue-resident macrophage enhancer landscapes are shaped by the local microenvironment. Cell 159, 1312-1326.

Lawrence, T., and Natoli, G. (2011). Transcriptional regulation of macrophage polarization: enabling diversity with identity. Nat Rev Immunol 11, 750-761.

Lee, J. H., Tammela, T., Hofree, M., Choi, J., Marjanovic, N. D., Han, S., Canner, D., Wu, K., Paschini, M., Bhang, D. H., et al. (2017). Anatomically and Functionally Distinct Lung Mesenchymal Populations Marked by Lgr5 and Lgr6. Cell 170, 1149-1163 e1112.

Luo, Y., Zhou, H., Krueger, J., Kaplan, C., Lee, S. H., Dolman, C., Markowitz, D., Wu, W., Liu, C., Reisfeld, R. A., et al. (2006). Targeting tumor-associated macrophages as a novel strategy against breast cancer. J Clin Invest 116, 2132-2141.

Mack, M., Schneider, M. A., Moll, C., Cihak, J., Bruhl, H., Ellwart, J. W., Hogarth, M. P., Stangassinger, M., and Schlondorff, D. (2005). Identification of antigen-capturing cells as basophils. J Immunol 174, 735-741.

Mackay, L. K., Rahimpour, A., Ma, J. Z., Collins, N., Stock, A. T., Hafon, M. L., Vega-Ramos, J., Lauzurica, P., Mueller, S. N., Stefanovic, T., et al. (2013). The developmental pathway for CD103(+)CD8+ tissue-resident memory T cells of skin. Nat Immunol 14, 1294-1301.

Macosko, E. Z., Basu, A., Satija, R., Nemesh, J., Shekhar, K., Goldman, M., Tirosh, I., Bialas, A.R., Kamitaki, N., Martersteck, E. M., et al. (2015). Highly Parallel Genome-wide Expression Profiling of Individual Cells Using Nanoliter Droplets. Cell 161, 1202-1214.

Mantovani, A., Schioppa, T., Porta, C., Allavena, P., and Sica, A. (2006). Role of tumor-associated macrophages in tumor progression and invasion. Cancer Metastasis Rev 25, 315-322.

Mantovani, A., Sica, A., Sozzani, S., Allavena, P., Vecchi, A., and Locati, M. (2004). The chemokine system in diverse forms of macrophage activation and polarization. Trends Immunol 25, 677-686.

Mantovani, A., Sozzani, S., Locati, M., Allavena, P., and Sica, A. (2002). Macrophage polarization: tumor-associated macrophages as a paradigm for polarized M2 mononuclear phagocytes. Trends Immunol 23, 549-555.

Marioni, J. C., and Arendt, D. (2017). How Single-Cell Genomics Is Changing Evolutionary and Developmental Biology. Annu Rev Cell Dev Biol 33, 537-553.

Marone, G., Galli, S. J., and Kitamura, Y. (2002). Probing the roles of mast cells and basophils in natural and acquired immunity, physiology and disease. Trends Immunol 23, 425-427.

Martins, R., Maier, J., Gorki, A. D., Huber, K. V., Sharif, 0., Starkl, P., Saluzzo, S., Quattrone, F., Gawish, R., Lakovits, K., et al. (2016). Heme drives hemolysis-induced susceptibility to infection via disruption of phagocyte functions. Nat Immunol 17, 1361-1372.

Mass, E., Ballesteros, I., Farlik, M., Halbritter, F., Gunther, P., Crozet, L., Jacome-Galarza, C. E., Handler, K., Klughammer, J., Kobayashi, Y., et al. (2016). Specification of tissue-resident macrophages during organogenesis. Science 353.

Matcovitch-Natan, O., Winter, D. R., Giladi, A., Vargas Aguilar, S., Spinrad, A., Sarrazin, S., Ben-Yehuda, H., David, E., Zelada Gonzalez, F., Perrin, P., et al. (2016). Microglia development follows a stepwise program to regulate brain homeostasis. Science 353, aad8670.

Medaglia, C., Giladi, A., Stoler-Barak, L., De Giovanni, M., Salame, T. M., Biram, A., David, E., Li, H., Iannacone, M., Shulman, Z., et al. (2017). Spatial reconstruction of immune niches by combining photoactivatable reporters and scRNA-seq. Science 358, 1622-1626.

Min, B., Prout, M., Hu-Li, J., Zhu, J., Jankovic, D., Morgan, E. S., Urban, J. F., Jr., Dvorak, A. M., Finkelman, F. D., LeGros, G., et al. (2004). Basophils produce IL-4 and accumulate in tissues after infection with a Th2-inducing parasite. J Exp Med 200, 507-517.

Morrisey, E. E., and Hogan, B. L. (2010). Preparing for the first breath: genetic and cellular mechanisms in lung development. Dev Cell 18, 8-23.

Mukai, K., Matsuoka, K., Taya, C., Suzuki, H., Yokozeki, H., Nishioka, K., Hirokawa, K., Etori, M., Yamashita, M., Kubota, T., et al. (2005). Basophils play a critical role in the development of IgE-mediated chronic allergic inflammation independently of T cells and mast cells. Immunity 23, 191-202.

Murphy, J., Summer, R., Wilson, A. A., Kotton, D. N., and Fine, A. (2008). The prolonged life-span of alveolar macrophages. Am J Respir Cell Mol Biol 38, 380-385.

Nabhan, A., Brownfield, D. G., Harbury, P. B., Krasnow, M. A., and Desai, T. J. (2018). Single-cell Wnt signaling niches maintain stemness of alveolar type 2 cells. Science.

Nowarski, R., Jackson, R., Gagliani, N., de Zoete, M. R., Palm, N. W., Bailis, W., Low, J.S., Harman, C.C., Graham, M., Elinav, E., et al. (2015). Epithelial IL-18 Equilibrium Controls Barrier Function in Colitis. Cell 163, 1444-1456.

Oh, K., Shen, T., Le Gros, G., and Min, B. (2007). Induction of Th2 type immunity in a mouse system reveals a novel immunoregulatory role of basophils. Blood 109, 2921-2927.

Ohmichi, H., Koshimizu, U., Matsumoto, K., and Nakamura, T. (1998). Hepatocyte growth factor (HGF) acts as a mesenchyme-derived morphogenic factor during fetal lung development. Development 125, 1315-1324.

Okabe, Y., and Medzhitov, R. (2014). Tissue-specific signals control reversible program of localization and functional polarization of macrophages. Cell 157, 832-844.

Okabe, Y., and Medzhitov, R. (2016). Tissue biology perspective on macrophages. Nat Immunol 17, 9-17.

Okubo, T., and Hogan, B.L. (2004). Hyperactive Wnt signaling changes the developmental potential of embryonic lung endoderm. J Biol 3, 11.

Panduro, M., Benoist, C., and Mathis, D. (2016). Tissue Tregs. Annu Rev Immunol 34, 609-633.

Paul, F., Arkin, Y., Giladi, A., Jaitin, D.A., Kenigsberg, E., Keren-Shaul, H., Winter, D., Lara-Astiaso, D., Gury, M., Weiner, A., et al. (2015). Transcriptional Heterogeneity and Lineage Commitment in Myeloid Progenitors. Cell 163, 1663-1677.

Perdiguero, E. G., and Geissmann, F. (2016). The development and maintenance of resident macrophages. Nat Immunol 17, 2-8.

Ramilowski, J. A., Goldberg, T., Harshbarger, J., Kloppmann, E., Lizio, M., Satagopam, V.P., Itoh, M., Kawaji, H., Carninci, P., Rost, B., et al. (2015). A draft network of ligand-receptor-mediated multicellular signalling in human. Nat Commun 6, 7866.

Rantakari, P., Jappinen, N., Lokka, E., Mokkala, E., Gerke, H., Peuhu, E., Ivaska, J., Elima, K., Auvinen, K., and Salmi, M. (2016). Fetal liver endothelium regulates the seeding of tissue-resident macrophages. Nature 538, 392-396.

Ries, C. H., Cannarile, M. A., Hoves, S., Benz, J., Wartha, K., Runza, V., Rey-Giraud, F., Pradel, L. P., Feuerhake, F., Klaman, I., et al. (2014). Targeting tumor-associated macrophages with anti-CSF-1R antibody reveals a strategy for cancer therapy. Cancer Cell 25, 846-859.

Rimoldi, M., Chieppa, M., Salucci, V., Avogadri, F., Sonzogni, A., Sampietro, G. M., Nespoli, A., Viale, G., Allavena, P., and Rescigno, M. (2005). Intestinal immune homeostasis is regulated by the crosstalk between epithelial cells and dendritic cells. Nat Immunol 6, 507-514.

Saluzzo, S., Gorki, A. D., Rana, B. M. J., Martins, R., Scanlon, S., Starkl, P., Lakovits, K., Hladik, A., Korosec, A., Sharif, O., et al. (2017). First-Breath-Induced Type 2 Pathways Shape the Lung Immune Environment. Cell Rep 18, 1893-1905.

Satija, R., Farrell, J. A., Gennert, D., Schier, A. F., and Regev, A. (2015). Spatial reconstruction of single-cell gene expression data. Nat Biotechnol 33, 495-502.

Schlitzer, A., McGovern, N., and Ginhoux, F. (2015). Dendritic cells and monocyte-derived cells: Two complementary and integrated functional systems. Semin Cell Dev Biol 41, 9-22.

Schmitz, J., Owyang, A., Oldham, E., Song, Y., Murphy, E., McClanahan, T. K., Zurawski, G., Moshrefi, M., Qin, J., Li, X., et al. (2005). IL-33, an interleukin-1-like cytokine that signals via the IL-1 receptor-related protein ST2 and induces T helper type 2-associated cytokines. Immunity 23, 479-490.

Schneider, C., Nobs, S. P., Kurrer, M., Rehrauer, H., Thiele, C., and Kopf, M. (2014). Induction of the nuclear receptor PPAR-gamma by the cytokine GM-CSF is critical for the differentiation of fetal monocytes into alveolar macrophages. Nat Immunol 15, 1026-1037.

Schneider, E., Petit-Bertron, A. F., Bricard, R., Levasseur, M., Ramadan, A., Girard, J. P., Herbelin, A., and Dy, M. (2009). IL-33 activates unprimed murine basophils directly in vitro and induces their in vivo expansion indirectly by promoting hematopoietic growth factor production. J Immunol 183, 3591-3597.

Sharif, O., Gawish, R., Warszawska, J. M., Martins, R., Lakovits, K., Hladik, A., Doninger, B., Brunner, J., Korosec, A., Schwarzenbacher, R. E., et al. (2014). The triggering receptor expressed on myeloid cells 2 inhibits complement component lq effector mechanisms and exerts detrimental effects during pneumococcal pneumonia. PLoS Pathog 10, e1004167.

Shen, H., Huang, G.J., and Gong, Y. W. (2003). Effect of transforming growth factor beta and bone morphogenetic proteins on rat hepatic stellate cell proliferation and trans-differentiation. World J Gastroenterol 9, 784-787.

Shibata, Y., Berclaz, P. Y., Chroneos, Z. C., Yoshida, M., Whitsett, J. A., and Trapnell, B. C. (2001). GM-CSF regulates alveolar macrophage differentiation and innate immunity in the lung through PU.1. Immunity 15, 557-567.

Snelgrove, R. J., Goulding, J., Didierlaurent, A. M., Lyonga, D., Vekaria, S., Edwards, L., Gwyer, E., Sedgwick, J. D., Barclay, A. N., and Hussell, T. (2008). A critical function for CD200 in lung immune homeostasis and the severity of influenza infection. Nat Immunol 9, 1074-1083.

Sojka, D. K., Plougastel-Douglas, B., Yang, L., Pak-Wittel, M. A., Artyomov, M. N., Ivanova, Y., Zhong, C., Chase, J. M., Rothman, P. B., Yu, J., et al. (2014). Tissue-resident natural killer (NK) cells are cell lineages distinct from thymic and conventional splenic NK cells. Elife 3, e01659.

Sokol, C. L., Chu, N. Q., Yu, S., Nish, S. A., Laufer, T. M., and Medzhitov, R. (2009). Basophils function as antigen-presenting cells for an allergen-induced T helper type 2 response. Nat Immunol 10, 713-720.

Sokol, C. L., and Medzhitov, R. (2010). Role of basophils in the initiation of Th2 responses. Curr Opin Immunol 22, 73-77.

Street, K., Risso, D., Fletcher, R. B., Das, D., Ngai, J., Yosef, N., Purdom, E., and Dudoit, S. (2017). Slingshot: Cell lineage and pseudotime inference for single-cell transcriptomics. bioRxiv.

Sullivan, B. M., Liang, H. E., Bando, J. K., Wu, D., Cheng, L. E., McKerrow, J. K., Allen, C. D., and Locksley, R. M. (2011). Genetic analysis of basophil function in vivo. Nat Immunol 12, 527-535.

Sullivan, B. M., and Locksley, R. M. (2009). Basophils: a nonredundant contributor to host immunity. Immunity 30, 12-20.

Sun, C. M., Hall, J. A., Blank, R. B., Bouladoux, N., Oukka, M., Mora, J. R., and Belkaid, Y. (2007). Small intestine lamina propria dendritic cells promote de novo generation of Foxp3 T reg cells via retinoic acid. J Exp Med 204, 1775-1785.

Tan, S. Y., and Krasnow, M. A. (2016). Developmental origin of lung macrophage diversity. Development 143, 1318-1327.

Tanay, A., and Regev, A. (2017). Scaling single-cell genomics from phenomenology to mechanism. Nature 541, 331-338.

Todd, E. M., Zhou, J. Y., Szasz, T. P., Deady, L. E., D'Angelo, J. A., Cheung, M. D., Kim, A. H., and Morley, S. C. (2016). Alveolar macrophage development in mice requires L-plastin for cellular localization in alveoli. Blood 128, 2785-2796.

Townsend, M. J., Fallon, P. G., Matthews, D. J., John, H. E., and McKenzie, A. N. (2000). T 1/ST2-deficient mice demonstrate the importance of T 1/ST2 in developing primary T helper cell type 2 responses. J Exp Med 191, 1069-1076.

Trapnell, B. C., and Whitsett, J. A. (2002). Gm-CSF regulates pulmonary surfactant homeostasis and alveolar macrophage-mediated innate host defense. Annu Rev Physiol 64, 775-802.

Treutlein, B., Brownfield, D. G., Wu, A. R., Neff, N. F., Mantalas, G. L., Espinoza, F. H., Desai, T. J., Krasnow, M. A., and Quake, S. R. (2014). Reconstructing lineage hierarchies of the distal lung epithelium using single-cell RNA-seq. Nature 509, 371-375.

Treutlein, B., Lee, Q. Y., Camp, J. G., Mall, M., Koh, W., Shariati, S. A., Sim, S., Neff, N. F., Skotheim, J. M., Wernig, M., et al. (2016). Dissecting direct reprogramming from fibroblast to neuron using single-cell RNA-seq. Nature 534, 391-395.

Tschopp, C. M., Spiegl, N., Didichenko, S., Lutmann, W., Julius, P., Virchow, J. C., Hack, C. E., and Dahinden, C. A. (2006). Granzyme B, a novel mediator of allergic inflammation: its induction and release in blood basophils and human asthma. Blood 108, 2290-2299.

Tsujimura, Y., Obata, K., Mukai, K., Shindou, H., Yoshida, M., Nishikado, H., Kawano, Y., Minegishi, Y., Shimizu, T., and Karasuyama, H. (2008). Basophils play a pivotal role in immunoglobulin-G-mediated but not immunoglobulin-E-mediated systemic anaphylaxis. Immunity 28, 581-589.

Varricchi, G., Raap, U., Rivellese, F., Marone, G., and Gibbs, B. F. (2018). Human mast cells and basophils-How are they similar how are they different? Immunol Rev 282, 8-34.

Voehringer, D., Liang, H. E., and Locksley, R. M. (2008). Homeostasis and effector function of lymphopenia-induced “memory-like” T cells in constitutively T cell-depleted mice. J Immunol 180, 4742-4753.

Whitsett, J. A., and Alenghat, T. (2015). Respiratory epithelial cells orchestrate pulmonary innate immunity. Nat Immunol 16, 27 -35.

Yanagita, K., Matsumoto, K., Sekiguchi, K., Ishibashi, H., Niho, Y., and Nakamura, T. (1993). Hepatocyte growth factor may act as a pulmotrophic factor on lung regeneration after acute lung injury. J Biol Chem 268, 21212-21217.

Yu, X., Buttgereit, A., Lelios, I., Utz, S. G., Cansever, D., Becher, B., and Greter, M. (2017). The Cytokine TGF-beta Promotes the Development and Homeostasis of Alveolar Macrophages. Immunity 47, 903-912 e904.

Yuk, C. M., Park, H. J., Kwon, B. I., Lah, S. J., Chang, J., Kim, J. Y., Lee, K. M., Park, S. H., Hong, S., and Lee, S. H. (2017). Basophil-derived IL-6 regulates TH17 cell differentiation and CD4 T cell immunity. Sci Rep 7, 41744.

Zepp, J. A., Zacharias, W. J., Frank, D. B., Cavanaugh, C. A., Zhou, S., Morley, M. P., and Morrisey, E. E. (2017). Distinct Mesenchymal Lineages and Niches Promote Epithelial Self-Renewal and Myofibrogenesis in the Lung. Cell 170, 1134-1148 e1110.

Zhou, X., Franklin, R. A., Adler, M., Jacox, J. B., Bailis, W., Shyer, J. A., Flavell, R. A., Mayo, A., Alon, U., and Medzhitov, R. (2018). Circuit Design Features of a Stable Two-Cell System. Cell 172, 744-757 e717.

What is claimed is:

1. A method of treating a disease or disorder that can benefit from increasing an M2/M1 macrophage ratio in a subject in need thereof, the method comprising:
(a) culturing basophils in the presence of IL33 and/or GM-SCF; and
(b) administering to the subject a therapeutically effective amount of said basophils following said culturing,
thereby treating the disease or disorder that can benefit from increasing an M2/M1 macrophage ratio in the subject.

2. The method of claim 1, wherein said basophils are blood circulating basophils or derived from the bone-marrow.

3. The method of claim 1, further comprises prior to (a):
(i) isolating said basophils from bone marrow or peripheral blood;
(ii) differentiating said basophils from said bone marrow or peripheral blood in the presence of IL-3 to as to obtain a differentiated culture;
(iii) isolating from said differentiated culture a cKIT- population.

4. The method of claim 3, wherein said (ii) is performed for 8-10 days in culture.

5. The method of claim 1, wherein said (a) is performed for up to 48 hours.

6. The method of claim 1, wherein said culturing is performed so as to achieve a lung basophil phenotype.

7. The method of claim 6, wherein said lung basophil phenotype comprises expression of growth factors and cytokines selected from the group consisting of Csf1, Il6, Il13, L1 cam, Il4, Ccl3, Ccl4, Ccl6, Ccl9 and Hgf, said expression being higher than in blood circulating basophils.

8. The method of claim 6, wherein said lung basophil phenotype comprises an expression signature of Il6, Il13, Cxcl2, Tnf, Osm and Ccl4.

9. The method of claim 6, wherein said lung basophil phenotype comprises an expression signature of Fcera1⁺, Il3ra⁺ (Cd123), Itaga2⁺ (Cd49b), Cd69⁺, Cd244⁺ (2B4), Itgam⁺ (Cd11b), Cd63⁺, Cd24a⁺, Cd200r3⁺, Il2ra⁶³⁰ , Il18rap⁺ and C3ar1⁺.

10. The method of claim 6, wherein said basophils are human.

11. The method of claim 10, wherein said basophils comprise an expression signature of Fcer1, Il13ra1, Itga2, Cd69, Cd244, Itgam, Cd63, Cd24, Il2ra, Il18rap and C3ar1.

12. The method of claim 1, wherein said basophils are autologous to the subject.

13. A method of treating a disease or disorder that can benefit from increasing an M2/M1 macrophage ratio in a subject in need thereof, the method comprising administering to the subject a therapeutically effective amount of a signaling molecule selected from the group consisting of IL6, IL13 and HGF, thereby treating the disease or disorder that can benefit from increasing an M2/M1 macrophage ratio in the subject.

14. The method of claim 1, wherein said therapeutically effective amount increases said M1/M2 macrophage ratio.

15. The method of claim 1, wherein said administering is in a local route of administration.

16. The method of claim 1, wherein said disease or disorder that can benefit from increasing an M2/M1 macrophage ratio is an inflammatory disease or an autoimmune disease.

17. The method of claim 1, wherein said disease or disorder that can benefit from increasing an M2/M1 macrophage ratio is a pulmonary disease.

18. The method of claim 1, wherein said M2/M1 macrophage comprises alveolar macrophages.

19. The method of claim 1, wherein said disease or disorder that can benefit from increasing an M2/M1 macrophage ratio is a chronic obstructive pulmonary disease (COPD).
